(19) United States
(12) Patent Application Publication
(10) Pub. No.: US 2010/0190778 A1
(43) Pub. Date: Jul. 29, 2010

(54) 3,4-DIHYDROBENZO[1,2,3]THIADIAZINE-1,1-DIOXIDE DERIVATIVES, PROCESS FOR PREPARATION THEREOF, MEDICAMENTS CONTAINING SAID DERIVATIVES AND THEIR USE

(75) Inventors: Marta Porcs-Makkay, Pomaz (HU); Gyula Lukacs, Budapest (HU); Gabor Kapus, Pecel (HU); Istvan Gacsalyi, Budapest (HU); Gyula Simig, Budapest (HU); Gyoergy Levay, Budakeszi (HU); Tibor Mezei, Budapest (HU); Miklos Vegh, Budapest (HU); Szabolcs Kertesz, Fot (HU); Jozsef Barkoczy, Budapest (HU); Csilla Leveleki, Budapest (HU); Laszlo Gabor Harsing, Budapest (HU)

Correspondence Address: KF ROSS PC, 5683 RIVERDALE AVENUE, SUITE 203 BOX 900, BRONX, NY, 10471-0900, US

(73) Assignee: EGIS GYOGYSZERGYAR NYILVANOSAN MUKODO RESZVENTARSASAG, BUDAPEST (HU)

(21) Appl. No.: 12/377,756

(22) PCT Filed: Aug. 13, 2007
(86) PCT No.: PCT/HU07/00072

§ 371 Date: Jul. 29, 2009

(30) Foreign Application Priority Data
Aug. 16, 2006 (HU) P0600651

(51) Int. Cl. A61K 31/5415; C07D 285/16; A61P 25/18; A61P 25/22; A61P 25/24

(52) U.S. Cl. 514/222.8; 544/11

(57) ABSTRACT

The present invention is related to new 3,4-dihydroberizo[1,2,3]thiadiazine-1,1-dioxide derivatives of the Formula (I), medicaments containing said new compounds, process for the preparation thereof and the use of said derivatives in the medicine. The compounds according to the present invention are suitable for the treatment or prevention of disorders of the central nervous system.

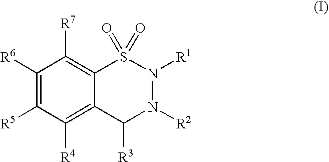

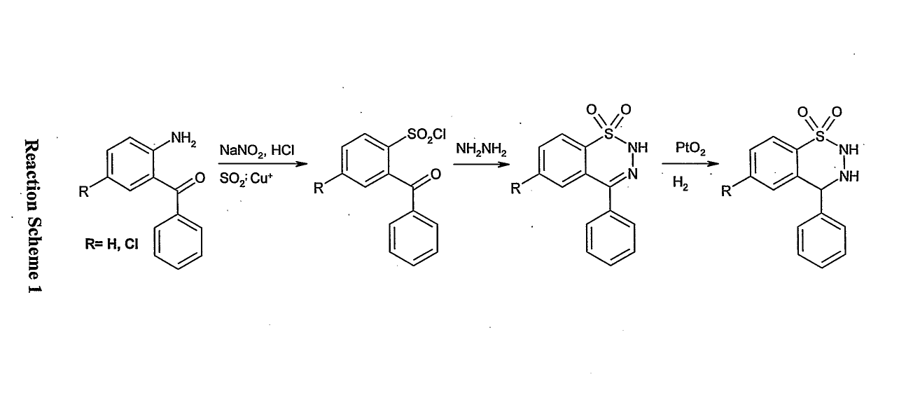

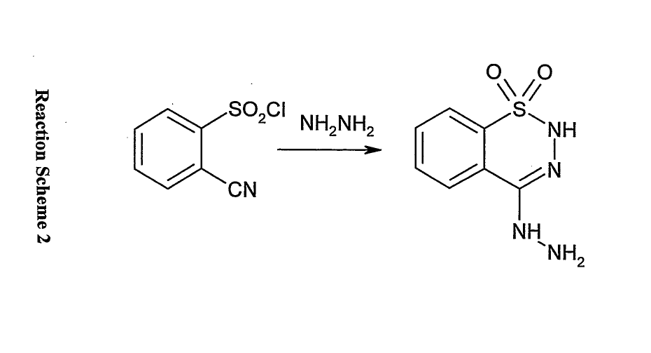

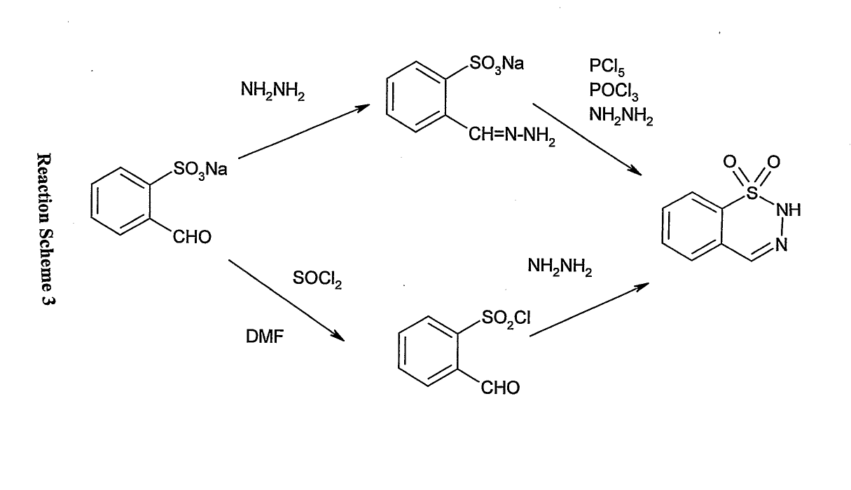

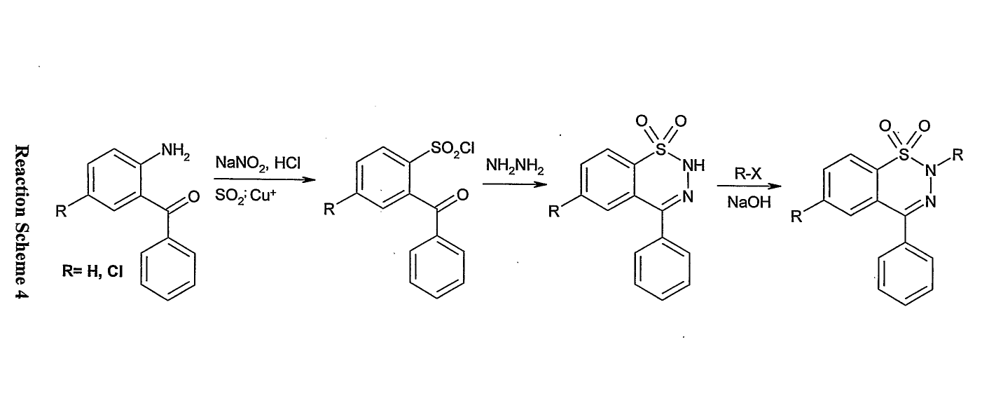

### TECHNICAL FIELD OF THE INVENTION

The present invention relates to new 3,4-dihydrobenzo[1,2,3]thiadiazine-1,1-dioxide derivatives of the general Formula (I)

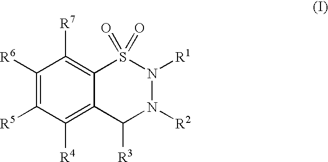

medicaments containing said derivatives, process for preparation of the compounds of the general Formula (I) and the use thereof in the medicine. The compounds of the general Formula (I) are useful in the prevention or treatment of diseases of the central nervous system.

The subject of the present invention is more specifically 3,4-dihydrobenzo[1,2,3]thiadiazine-1,1-dioxide derivatives of the general Formula (I), wherein

R¹, R², R³ represents independently from each other hydrogen, a straight- or branched-chain alkyl group having 1 to 4 carbon atoms,
R⁴, R⁵, R⁶ and R⁷ represents independently hydrogen, halogen, a straight- or branched-chain alkyl group having 1 to 4 carbon atoms, an alkoxy group containing a straight- or branched-chain alkyl-group having 1 to 4 carbon atoms,
and the stereoisomers of the compounds of the general Formula (I) and the mixtures thereof.

### TECHNICAL BACKGROUND OF THE INVENTION

The analogones of the 3,4-dihydrobenzo[1,2,3]thiadiazine-1,1-dioxide compounds of the general Formula (I) have been disclosed for the first time in 1968 (J. B. Wright, J. Het. Chem. 1968, 5, 453-459). In U.S. Pat. No. 3,407,197, analogons containing phenyl-group in position 4 are described, which exhibit disinfectant, herbicide and pesticide effect. The compounds were prepared by the hydrogenation of 4-phenylbenzo[1,2,3]thiadiazine-1,1-dioxide or 4-phenyl-6-chlorobenzo[1,2,3]thiadiazine-1,1-dioxide in acetic acid solvent in the presence of platinum(IV)oxide catalyst (Reaction Scheme 1).

The starting materials in the synthesis of the compounds of the general Formula (I) according to the present invention are benzo[1,2,3]thiadiazine-1,1-dioxide derivatives, which are known from the prior art (E. Schrader, J. Prakt. Chem. 1918, 96, 180-185; P. Schmidt, K. Eichenberger, M. Wilhelm, Helv. Chim. Acta 1962, 45, 996-999). Said starting compounds can be prepared for example by reacting 2-cyano-benzenesulfonylchloride with hydrazine, which yields 3-hydrazino-benzo[1,2,3]thiadiazine-1,1-dioxide derivatives (Reaction Scheme 2).

The benzo[1,2,3]thiadiazine-1,1-dioxide structural unit serving as a starting compound in the synthesis of the compound of the general Formula (I) can be prepared by two methods starting from sodium o-formyl-benzenesulfonate (J. F. King, A. Hawson, D. M. Deaken, J. Komery, J. C. S. Chem. Comm. 1969, 33-34; J. F. King, A. H. Huston, J. Komery, D. M. Deaken, D. R. K. Harding, Can. J. Chem 1971, 49, 936-942). The two reaction routes are demonstrated in Reaction Scheme 3.

The first process comprises reacting sodium o-formyl-benzenesulfonate with thionylchloride and reacting the thus obtained o-formyl-benzenesulfonylchloride with hydrazine, yielding benzo[1,2,3]thiadiazine-1,1-dioxide.

According to the second process, the sodium salt of o-formyl-benzenesulfonic acid is reacted with hydrazine and the thus obtained hydrazide is subjected to ring closure in the presence of phosphorous pentachloride or phosphorous oxychloride and hydrazine, yielding benzo[1,2,3]thiadiazine-1,1-dioxide.

4-phenyl-benzo[1,2,3]thiadiazine-1,1-dioxide derivatives can be prepared according to the state of the art starting from o-amino-benzophenone. 0-amino-benzophenone is converted into a diazonium salt, which is subsequently transformed into the corresponding sulfochloride by reacting the diazonium salt with sulfur dioxide in the presence of copper(I)-salts. The sulfochloride is subsequently converted into 4-phenyl-benzo[1,2,3]thiadiazine-1,1-dioxide using one of the two methods described above (J. B. Wright, J. Het. Chem 1968, 5, 453-459). The synthetic route is demonstrated in Reaction Scheme 4.

Due to the accelerated technical-scientific development and social changes, a human living in the civilized environment is exposed to a number of stress sources. Under such conditions, disorders or diseases belonging to the group of anxiety disorders can develop due to the endured stress.

Anxiety is a characteristic symptom of the central nervous system, which can be manifested in the form of medical, psychiatric, traumatic, surgical diseases, disorders or states as well. Medicines most often used in the treatment of various anxiety disorders are the so-called benzodiazepine-type agents, i.e. diazepam, chlorodiazepoxide, alprazolam.

Although there are several pharmaceutically active ingredients available for the treatment of anxiety disorders and the states developing as a result of such disorders, said active ingredients exhibit several non-desirable side effects having disadvantageous effect on the lifestyle and life quality. Furthermore, it is known fact according to the state of the art that several diseases of the central nervous system can be treated most effectively by combination therapy. Thus, there exists a continuous need for the development of new pharmaceutically active ingredients which are suitable for the use in anxiolytic medicaments.

### SUMMARY OF THE INVENTION

The objective of our research was to develop new pharmaceutically active ingredients suitable for the treatment or prevention of anxiety disorders, i.e. general anxiety disorder, panic disorder, agoraphobia, social phobia, other types of phobias, posttraumatic stress disorder and for the prevention or treatment of the symptoms of the diseases of the central nervous system accompanied by the different forms of anxiety disorders.

The above objective has been solved by the present invention.

The present invention is based on the surprising recognition that 3,4-dihydrobenzo[1,2,3]thiadiazine-1,1-dioxides of the Formula (I) wherein

R¹, R², R³ represent independently hydrogen or a straight- or branched-chain alkyl-group having 1 to 4 carbon atoms,
R⁴, R⁵, R⁶ és R⁷ represent independently hydrogen, halogen, straight- or branched-chain alkyl-group having 1 to 4 carbon atoms or an alkoxy group containing a branched- or straight-chain alkyl-group having 1 to 4 carbon atoms,
possess significant anxiolytic activity and due to this activity, said compounds of the general Formula (I) are suitable form the treatment or prevention of diseases, disorders or states belonging to the group of anxiety disorders, e.g. generalized anxiety disorder, panic disorder, agoraphobia, social phobia, other types of phobias, posttraumatic stress disorder and in the treatment or prevention of all disorders and diseases of the central nervous system, which manifest themselves in or accompanied by the symptoms of anxiety, e.g. attention-deficit hyperactivity disorder, disturbance of adaptation caused by stress, posttraumatic stress disorder, anorexia nervosa, bulimia nervosa, insomnia, parasomnia, compulsive disorders including obsessive-compulsive disorder, disturbances of sexual function, symptoms of drug withdrawal or use with respect to alcohol, caffeine, drugs of abuse, hypnotics, sedatives or doping agents. The compounds of the general Formula (I) are new.

The anxiolytic effect of the compounds of the general Formula (I) is especially surprising, since it is entirely unrelated to the already known diuretic, disinfectant, insecticide or herbicide effect of 3,4-dihydrobenzo[1,2,3]thiadiazine-1,1-dioxide derivatives known from the prior art. Although there is a disclosure about the central nervous system effects of certain benzo[1,2,3]thiadiazine-1,1-dioxide derivatives in the prior art, the prior art is silent about both the range of activity and the strength of said effect.

### DETAILED DESCRIPTION OF THE INVENTION

According to the first aspect of the present invention, there are provided 3,4-dihydrobenzo[1,2,3]thiadiazine-1,1-dioxide derivatives of the general Formula (I), wherein

R¹, R², R³ represents independently from each other hydrogen or a straight- or branched-chain alkyl group having 1 to 4 carbon atoms,
R⁴, R⁵, R⁶ és R⁷ each represents hydrogen, halogen, straight- or branched-chain alkyl group having 1 to 4 carbon atoms or an alkoxy group containing a straight or branched-chain alkyl group having 1 to 4 carbon atoms,
and the stereoisomers of the compound of the general Formula (I) and the mixtures thereof.

In the present application, the meaning of “halogen” is fluorine, bromine, chlorine or iodine.

The expression “straight- or branched chain alkyl-group comprising 1 to 4 carbon atoms” means a saturated hydrocarbon group comprising 1 to 4 carbon atoms, e.g. methyl, ethyl, n-propyl-, izopropyl-, n-butyl-, sec-butyl-, tert-butyl- or isobutyl-group.

The expression “alkoxy group comprising 1 to 4 carbon atoms” means an alkoxy group wherein the alkyl group is a straight- or branched-chain alkyl having 1 to 4 carbon atoms according to the above definition.

According to a further aspect of the present invention, there is provided a process for the preparation of 3,4-dihydrobenzo[1,2,3]thiadiazine-1,1-dioxide derivatives of the general Formula (I).

The first process variant suitable for the preparation of the compounds of the general Formula (I) comprises reducing a benzo[1,2,3]thiadiazine-1,1-dioxide derivative of the general Formula (II)

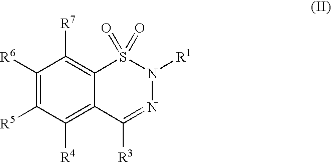

According to the second process variant, a compound of the general Formula (II) is converted into the corresponding 3,4-dihydrobenzo[1,2,3]thiadiazin-1,1-dioxide of the general Formula (I) and thereafter transforming the compound of the general Formula (I) thus obtained into a different compound of the general Formula (I).

The third process variant developed for the preparation of the 3,4-dihydrobenzo[1,2,3]thiadiazine-1,1-dioxide derivatives of the general Formula (I) comprises reducing a compound of the general Formula (III).

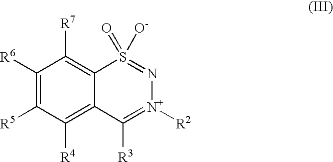

Those 3,4-dihydrobenzo[1,2,3]thiadiazine-1,1-dioxide derivatives of the general Formula (I) wherein R¹ represents hydrogen or a straight- or branched-chain alkyl group having 1 to 4 carbon atoms, R² represents hydrogen and the meaning of R³, R⁴, R⁵, R⁶, R⁷ is as defined above, can be prepared by reducing a suitable compound of the general Formula (II) wherein R¹ represents hydrogen or a straight- or branched-chain alkyl-group and the meaning of R³, R⁴, R⁵, R⁶, R⁷ is the above, by using analogous methods to those already known from the prior art (Houben-Weyl: Reduktion I, 4/1c, p. 240, 271; J. B. Wright, J. Het. Chem. 1968, 5, 453-459). The reduction can be carried out by heterogeneous phase catalysis using a noble metal catalyst, preferably platinum(IV)oxide using a pressurized vessel under hydrogen pressure in an organic solvent, preferably acetic acid.

The 3,4-dihydrobenzo[1,2,3]thiadiazine-1,1-dioxide derivatives of the Formula (I), wherein R¹ represents a straight- or branched-chain alkyl-group, R² is hydrogen and the meaning of R³, R⁴, R⁵, R⁶, R⁷ is as defined above, can also be prepared by alkylating a compound of the general Formula (I), wherein R¹ and R² represents independently from each other hydrogen and the meaning of R³, R⁴, R⁵, R⁶, R⁷ is the above, in analogous manner to the methods known for the prior art (Houben-Weyl: Amine, XI/1, p. 98; J. B. Wright, J. Het. Chem. 1968, 5, 453-459). Preferably, the alkylation is carried out using alkyl halogenides using an organic solvent in the presence of an acid-binding reagent.

In place of the acid-binding agent, an inorganic or an organic base, preferably potassium-tent-butylate, sodium hydride or triethylamine can be used.

Suitable organic solvents are preferably polar aprotic solvents, e.g. N,N-dimethylformamide or tetrahydrofurane.

The preparation of 3,4-dihydrobenzo[1,2,3]thiadiazine-1,1-dioxide derivatives of the general Formula (I), wherein R¹ represents hydrogen or a straight- or branched-chain alkyl group having 1 to 4 carbon atoms, R² represents a straight- or branched-chain alkyl group having 1 to 4 carbon atoms and the meaning of R³, R⁴, R⁵, R⁶, R⁷ is as defined above, can be prepared by reductive alkylation. The reductive alkylation reaction comprises reacting a compound of the general Formula (I), wherein R¹ represents hydrogen or a straight- or branched-chain alkyl group comprising 1 to 4 carbon atoms R² is hydrogen and the meaning of R³, R⁴, R⁵, R⁶, R⁷ is as defined above with a suitable aldehyde or ketone (Houben-Weyl: Reduktion I, 4/1c, p. 411) using a catalyst, preferably palladium-charcoal or platinum(IV)oxide in an organic solvent, preferably in tetrahydrofurane in the presence of acetic acid and carrying out the reaction in a pressurized vessel under hydrogen pressure.

The preparation of 3,4-dihydrobenzo[1,2,3]thiadiazine-1,1-dioxide of the general Formula (I) wherein R¹ is hydrogen or a straight- or branched-chain alkyl-group having 1 to 4 carbon atoms, R² represents a straight- or branched-chain alkyl group comprising 1 to 4 carbon atoms and the meaning of R³, R⁴, R⁵, R⁶, R⁷ is as defined above, can be performed by the so-called “one-pot” synthesis method. In this type of synthesis, a compound of the general Formula (II) wherein R¹ represents hydrogen or a straight- or branched-chain alkyl and the meaning of R³, R⁴, R⁵, R⁶, R⁷ is as defined above, can be used as starting material. In the first reaction step, the double bond is saturated (reduced), and subsequently the thus obtained product is subjected to reductive alkylation using an aliphatic aldehyde or ketone (Houben-Weyl: Reduktion I, 4/1c, p. 411). The reductive alkylation is performed using preferably a palladium-charcoal or platinum(IV)oxide catalyst in an organic solvent, in the presence of acetic acid under hydrogen pressure. Preferably, the reaction is carried out in tetrahydrofurane solvent.

Those compounds of the general Formula (I), wherein R¹ and R³ each represents hydrogen, R² represents a straight- or branched-chain alkyl group having 1 to 4 carbon atoms and the meaning of R⁴, R⁵, R⁶, R⁷ is the above, can be prepared starting from those compounds of the general Formula (III) wherein R² represents a straight- or branched-chain alkyl-group having 1 to 4 carbon atoms and R³ represents hydrogen and the meaning of R⁴, R⁵, R⁶, R⁷ is as defined above, by catalytic reduction under hydrogen pressure in the presence of platinum(IV)oxide catalyst or by reducing said compound of the general Formula (III) with sodium borohydride (Houben-Weyl: Reduktion II, 4/1d, p. 347).

The compounds of the general Formula (III) wherein the meaning of R², R³, R⁴, R⁵, R⁶, R⁷ is as defined above, can be prepared by alkylation from those compounds of the general Formula (II) wherein R¹ and R³ each represents hydrogen and the meaning of R⁴, R⁵, R⁶, R⁷ is the above, by the methods known from the prior art (A. N. Kost, K. V. Grabliauskas, J. Prakt. Chem. 1970, 312, 542). The alkylation reaction is performed preferably by using an alkyl halogenide in the presence of a base, preferably in the presence of sodium hydride in an organic solvent, preferably, tetrahydrofurane.

Those 3,4-dihydrobenzo[1,2,3]thiadiazine-1,1-dioxide derivatives of the general Formula (I), wherein R¹ and R² represents independently a straight- or branched-chain alkyl group having 1 to 4 carbon atoms and the meaning of R³, R⁴, R⁵, R⁶, R⁷ is as defined above, can be prepared from those compounds of the general Formula (I) wherein R¹ represents hydrogen, R² is a straight- or branched-chain alkyl group having 1 to 4 carbon atoms and the meaning of R³, R⁴, R⁵, R⁶, R⁷ is the above, by alkylation in analogous manner to the methods known from the prior art (Houben-Weyl: Amine, XI/1, p. 98). The alkylation is carried out preferably using an alkyl halogenide as reactant, in the presence of an acid-binding reagent, preferably potassium-tent-butylate or sodium hydride, using an organic solvent of dipolar aprotic type, e.g. N,N-dimethylformamide or tetrahydrofurane.

Pharmacological activity of the compounds of the general Formula (I) has been evaluated in elevated maze test in the rat.

The tests were carried out according to the method of S. Fellow and coworkers (J. Neurosci. Methods. 1985, 14, 149-167). During the test, a wooden-floor cross elevated by 50 cm from the ground was used. The length and the width of the arms of the cross were 100 and 15 cm, respectively. Two opposite arms of the cross are fitted at the ends and both sides with black plexiglass wall of 40 cm height (closed arms). The central section of the cross measuring 15 by 15 cm is open. The other two opposite arms of the cross are not provided with walls (open arms).

The tests were carried out in male Sprague-Dawley rats weighing 200 to 220 g. After the 60-min pretreatment time (per os treatment), animals were placed in the center of the maze. During the 5-min measurement time, four variables were recorded: the time spent in the open arms; the time spent in the closed arms; the number of entries into the open arms; the number of entries into the closed arms.

Animals normally avoid the open and light parts of the maze, thereby spending little time therein only. The anxiolytic effect of the compounds of the general Formula (I) is indicated by the increase in the time spent (in seconds) in the open arms and/or by the increase in the number of the entries into the open compartments of the maze. The minimum effective dose (MED) was determined for each tested compounds regarding the time spent in the open arms and the number of entries into the open arms. During the test, the known anxiolytic compound I-methyl-5-phenyl-7-chloro-1,3-dihydro-2H-1,4-benzodiazepin-2-one (INN: diazepam) of the Formula (IV)

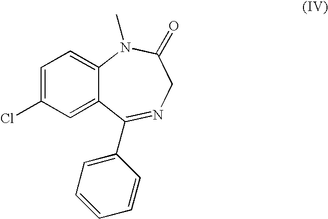

was used. Treatment of the animals was carried out orally with the doses 0.1, 0.3, 1.0, 3.0 for diazepam and 0.01, 0.1, 1.0 mg/kg for the compounds of the general Formula (I).

TABLE 1
Anxiolytic effect of the compounds of the general
Formula (I) in elevated maze test

| No. of | MED |
|---|---|
| Example | (mg/kg p.o.) |
| Diazepam | 1.00 |
| 1 | 0.3 |
| 3 | 0.01 |
| 5 | 0.01 |
| 16 | 0.01 |
| 20 | 0.01 |

The effect of the test compound resulted in the increase of the period of time spent in the open part and the number of the entries therein (Table 1). These indicate that 3,4-dihydrobenzo[1,2,3]thiadiazine-1,1-dioxide derivatives of the general Formula (I) possess significant anxiolytic activity.

From the results of the above experiments, it has been concluded that the compounds of the general Formula (I) possess entirely unexpected and surprisingly significant anxiolytic effect in a rodent behavioural model. Thus the compounds of the general Formula (I) are suitable for the treatment or prevention of diseases, disorders or states belonging to the group of anxiolytic disorders, i.e. generalized anxiety disorder, panic disorder, agoraphobia, social phobia, other types of phobias, posttraumatic stress disorder and any other disorders of the central nervous system, which are accompanied by the symptoms of anxiety. Such disorders include attention-deficit hyperactivity disorder, stress-related adaptation disorder, posttraumatic stress disorder, insomnia, parasomnia, compulsive disorders including obsessive-compulsive disorder, disturbances of the sexual function anorexia, bulimia, and symptoms of the use or withdrawal of drugs or chemical agents, including but not limited to the withdrawal of alcohol, caffeine, nicotine, sedatives, narcotics, doping agents or drugs of abuse.

The above-mentioned anxiolytic effect is entirely unexpected since the compounds chemically similar to those of the general Formula (I) were known as diuretic, herbicide or pesticide compounds. The anxiolytic effect is not related to the activity of the analogous compounds known from the prior art in any way.

According to a further aspect of the present invention, there are provided medicaments which contain one or more compound(s) of the general Formula (I) and one or more known vehicle(s) or auxiliary agent(s).

The proportion of the active ingredient of the general Formula (I) in the medicaments according to the present invention is generally between 0.1 and 95% by weight, preferably between 5 and 75% by weight.

The medicaments according to the present invention can be administered orally (e.g. powders, tablets, coated tablets, capsules, microcapsules, granules, pellets, dragee, solutions, suspensions or emulsions), parenterally (e.g. in the form of intravenous, intramuscular, subcutaneous or intraperitoneal injections or as infusions), rectally (e.g. as suppositories), transdermally (e.g. as patches), in the form of implants or locally (e.g. creams, ointments, patches). The medicaments according to the present invention can be prepared by the methods of pharmaceutical technology known from the prior art.

Medicaments containing the compounds of the general Formula (I) as active ingredients suitable for oral administration can also contain filling agents or vehicles (e.g. lactose, glucose, starch, calcium phosphate, microcrystalline cellulose), binders (gelatine, sorbitol, polyvinylpyrollidone), disintegrants (e.g. croscarmellose, sodium carboxymethylcellulose, crospovidone), tabletting aids (e.g. magnesium stearate, talc, polyethyleneglycol, silicic acid, silicon dioxide) or surfactants (e.g. sodium lauryl sulfate).

Liquid medicaments suitable for oral administration containing a compound of the general Formula (I) can be prepared in the form of solutions, suspensions or emulsions and can contain suspending agents (e.g. gelatine, carboxymethylcellulose), emulsifying agents (e.g. sorbitane monooleate), solvents (e.g. water, oils, glycerol, propylene glycol, ethanol), buffers (e.g. acetate, phosphate, citrate buffer) or stabilizing agents (e.g. methyl-4-hydroxybenzoate).

Medicaments suitable for parenteral administration containing a compounds of the general Formula (I) are usually sterile isotonic solutions, which can contain pH-adjusting agents and conservants besides the solvent.

The semisolid medicaments containing a compound of the general Formula (I), e.g. suppositories contain the pharmaceutically active ingredient homogeneously dispersed in the suppository base (.e.g. polyethylene glycol, cocoa butter).

The medicaments according to the present invention containing one or more compound(s) of the general Formula (I) as active ingredient(s) can be prepared in the form of modified-, extended-, or controlled-release preparation. Thus the release of the active compound of the general Formula (I) can be provided according to predetermined time function and thus a long-lasting therapeutical effect can be obtained or the frequency of the administration can be decreased. Such modified-, extended- or controlled-release medicaments can be prepared according to methods known from the prior art.

According to a further aspect of the present invention, there is provided a process for the preparation of medicaments containing one or more compound(s) of the general Formula (I) which comprises admixing one or more compound(s) of the general Formula (I) with a pharmaceutically acceptable carrier and if desired, with further auxiliary agents and transforming the thus obtained mixture into a pharmaceutical dosage form. The vehicles and auxiliary agents used in the pharmaceutical technology are known from the prior art (Remington's Pharmaceutical Sciences, Edition 18, Mack Publishing Co., Easton, USA, 1990).

Medicaments containing a compound of the general Formula (I) as active ingredient generally contain the active ingredient in the form of dosage units.

A further aspect of the present invention is the use of 3,4-dihydrobenzo[1,2,3]thiadiazine-1,1-dioxide derivatives of the Formula (I) for the treatment or prevention of anxiolytic disorders, i.e. generalized anxiety disorder, panic disorder, agoraphobia, social phobia, other types of phobias, posttraumatic stress disorder and any other disorders of the central nervous system, which are accompanied by the symptoms of anxiety including attention-deficit hyperactivity disorder, stress-related adaptation disorder, posttraumatic stress disorder, insomnia, parasomnia, compulsive disorders including obsessive-compulsive disorders, disturbances of the sexual function anorexia, bulimia, attention-deficient hyperactivity, obsessive-compulsive disorder, disturbances of the sexual function and symptoms of the withdrawal of drugs or chemical agents, including but not limited to the withdrawal of alcohol, caffeine, nicotine, sedatives, narcotics, doping agents or drugs of abuse.

According to another aspect of the present invention, there is provided a method for the treatment or prevention of diseases, disorders or states belonging to the group of anxiolytic disorders, i.e. generalized anxiety disorder, panic disorder, agoraphobia, social phobia, other types of phobias, posttraumatic stress disorder and any other disorders of the central nervous system, which are accompanied by the symptoms of anxiety. Such disorders include attention-deficit hyperactivity disorder, stress-related adaptation disorder, posttraumatic stress disorder, insomnia, parasomnia, compulsive disorders including obsessive-compulsive disorders, disturbances of the sexual function, anorexia, bulimia, attention-deficient hyperactivity, obsessive-compulsive disorder, disturbances of the sexual function and symptoms of the withdrawal of drugs or chemical agents, including but not limited to the withdrawal of alcohol, caffeine, nicotine, sedatives, narcotics, doping agents or drugs of abuse, which comprises administering to a patient in need of such treatment a therapeutically effective amount of a 3,4-dihydrobenzo[1,2,3]thiadiazine-1,1-dioxide derivative according to the present invention.

The applicable dose depends on several factors including the type and severity of the disease to be treated, the age, physiological status, body weight of the patient and other forms of therapy simultaneously used. The applicable dose should be determined by a physician.

The invention is further demonstrated in the following examples without limiting the invention to said examples.

### Example 1

### 7-chloro-4-methyl-3,4-dihydrobenzo[1,2,3]thiadiazine-1,1-dioxide

7-chloro-4-methylbenzo[1,2,3]thiadiazine-1,1-dioxide (16.3 g; 0.071 mol) is dissolved in acetic acid (200 ml) and after addition of platinum(IV)-oxide catalyst, (0.5 g) hydrogenated at 10 bar hydrogen pressure. After the calculated amount of hydrogen is used up, the catalyst is filtered off and the filtrate is evaporated.

Yield, 15.2 g, white crystals (92%)

Melting point, 155-156° C.

IR (KBr): 3182 (NH); 1328; 1166 (S═O) cm⁻¹.

¹HNMR (DMSO, 400 MHz): 8.69 (1H, s); 7.78 (1H, d, J=2.3 Hz); 7.64 (1H, dd, J=2.3; 8.5 Hz); 7.48 (1H, dd, J=0.5; 8.5 Hz); 4.22 (1H, q, J=6.7 Hz); 3.9 (1H, s); 1.35 (3H, d, J=6.7 Hz) ppm.

¹³CNMR: (DMSO, 400 MHz): 140.5; 138.3; 132.0; 131.9; 129.4; 123.3; 51.4; 19.0 ppm.

Elemental analysis [calculated on the basis of the Formula a C₈H₉ClN₂O₂S (232.69)]:

Calculated: C, 41.29; H, 3.90; Cl 15.24; N 12.04; S 13.78%

Measured: C, 41.45; H, 3.54; Cl 15.53; N 11.81; S 13.44%

### Example 2

### 4-Ethyl-7-chloro-3,4-dihydrobenzo[1,2,3]thiadiazine-1,1-dioxide

The title compound is produced according to the procedure of Example 1 starting from 4-ethyl-7-chlorobenzo[1,2,3]thiadiazine-1,1-dioxide (1.0 g; 0.0041 mol) using platinum(IV)-oxide (0.1 g).

Yield, 0.95 g, white crystals (94%)

Melting point, 128-130° C. (hexane-ethylacetate 1:1).

IR (KBr): 3339; 3201 (NH); 1311; 1170 (S═O) cm⁻¹.

¹HNMR. (CDCl₃, 400 MHz): 7.78 (1H, d, J=2.0 Hz); 7.46 (1H, dd, J=2.1; 8.5 Hz); 7.17 (1H, d, J=8.5 Hz); 6.0 (1H, s); 4.85 (1H, s); 3.99 (1H, t, J=3.8 Hz); 1.95-1.80 (2H, m); 1.05 (3H, t, J=7.4 Hz) ppm.

¹³CNMR (CDCl₃, 400 MHz): 137.9; 137.3; 133.6; 132.4; 128.3; 123.8; 57.5; 27.0; 10.3 ppm.

Elemental analysis [calculated on the basis of the Formula a C₉H₁₁ClN₂O₂S (246.72)]:

Calculated: C, 43.82; H, 4.49; Cl 14.37; N 11.35; S 13.00%

Measured: C, 43.68; H, 4.52; Cl 14.24; N 11.27; S 13.07%

### Example 3

### 7,8-Dichloro-4-methyl-3,4-dihydrobenzo[1,2,3]thiadiazine-1,1-dioxide

The title compound is produced according to the procedure of Example 1 starting from 7,8-dichloro-4-methylbenzo[1,2,3]thiadiazine-1,1-dioxide (3.0 g; 0.011 mol) using platinum(IV)-oxide (0.3 g).

Yield, 2.9 g white crystals (99%)

Melting point, 261-263° C. (ethanol)

IR (KBr): 3311 (NH); 1335, 1164 (S═O) cm⁻¹.

¹HNMR (DMSO, 400 MHz): 8.79 (1H, s); 7.83 (1H, d, J=8.6 Hz); 7.48 (1H, d, J=8.6 Hz); 6.08 (1H, s); 4.27 (1H, dq, J=2.1; 6.5 Hz); 1.35 (3H, d, J=6.7 Hz) ppm.

¹³CNMR (DMSO, 400 MHz): 143.6; 136.9; 132.9; 131.7; 128.2; 127.7; 52.5; 19.1 ppm.

Elemental analysis [calculated on the basis of the Formula C₈H₈Cl₂N₂O₂S (267.14)]:

Calculated: C, 35.97; H, 3.02; Cl 26.54; N 10.49; S 12.00%

Measured: C, 35.97; H, 3.04; Cl 26.69; N 10.36; S 11.84%

### Example 4

### 7,8-Dichloro-4-ethyl-3,4-dihydrobenzo[1,2,3]thiadiazine-1,1-dioxide

The title compound is prepared according to the procedure of Example 1 with the modification that the starting compound is 7,8-dichloro-4-ethylbenzo[1,2,3]thiadiazine-1,1-dioxide (5.0 g; 0.018 mol) and that platinum(IV)-oxide (0.5 g) is used.

Yield, 4.8 g, white crystals (89%)

Melting point, 222-224° C. (acetone)

IR (KBr): 3286; 3188 (NH), 1334, 1.160 (S═O) cm⁻¹.

¹HNMR (DMSO, 200 MHz): 8.76 (1H, s); 7.82 (1H, d, J=8.8 Hz); 7.47 (1H, dd, J=0.7; 8.8 Hz); 6.07 (1H, d, J=2.6 Hz); 4.08-4.03 (1H, m); 1.98-1.68 (2H, m); 0.86 (3H, t, J=7.3 Hz) ppm.

¹³CNMR (DMSO, 200 MHz): 142.6; 137.2; 132.8; 131.5; 128.1; 127.9; 57.2; 25.8; 9.9 ppm.

Elemental analysis [calculated on the basis of the Formula a C₉H₁₀Cl₂N₂O₂S (281.16)]:

Calculated: C, 38.45; H, 3.59; Cl 25.22; N 9.96; S 11.40%

Measured: C, 38.60; H, 3.63; Cl 25.06; N 9.90; S 11.10%

### Example 5

### 7,8-Dichloro-4,5-dimethyl-3,4-dihydrobenzo[1,2,3]thiadiazine-1,1-dioxide

The title compound is prepared according to the procedure of Example 1 starting from 7,8-dichloro-4,5-dimethylbenzo[1,2,3]thiadiazine-1,1-dioxide (2.15 g; 0.0077 mol) and using platinum(IV)-oxide (0.1 g).

Yield, 1.33 g, white crystals (61%).

Melting point, 197-199° C. (ethanol).

IR (KBr): 3336; 3215 (NH); 1321, 1180 (S═O) cm⁻¹.

¹HNMR (DMSO, 400 MHz): 8.97 (1H, d, J=4.7 Hz); 7.72 (1H, s); 6.17 (1H, dd, J=2.4; 4.7 Hz); 4.09 (1H, dq, J=2.4; 6.8 Hz); 2.30 (3H, s); 1.40 (3H, d, J=6.8 Hz) ppm.

¹³CNMR (DMSO, 400 MHz): 142.4; 136.7; 136.1; 134.5; 131.2; 125.5; 49.7; 18.8; 18.0 ppm.

Elemental analysis [calculated on the basis of the Formula a C₉H₁₀Cl₂N₂O₂S (281.16)]:

Calculated: C, 38.45; H, 3.59; Cl 25.22; N 9.96; S 11.40%

Measured: C, 38.55; H, 3.55; Cl 25.32; N 9.79; S 11.27%

### Example 6

### 7,8-Dichloro-4,6-dimethyl-3,4-dihydrobenzo[1,2,3]thiadiazine-1,1-dioxide

The title compound is prepared according to the procedure of Example 1 using 7,8-dichloro-4,6-dimethylbenzo[1,2,3]thiadiazine-1,1-dioxide (2.29 g; 0.0082 mol) and platinum(IV)oxide (0.1 g).

Yield, 2.0 g, white crystals (87%)

Melting point, 253-254° C. (ethanol)

IR (KBr): 3317, 3073 (NH); 1333, 1171 (S═O) cm⁻¹.

¹HNMR (DMSO, 400 MHz): 8.71 (1H, s); 7.48 (1H, s); 6.03 (1H, s); 4.22 (1H, m); 2.43 (3H, s); 1.35 (3H, d, J=6.7 Hz) ppm.

¹³CNMR (DMSO, 400 MHz): 142.4; 141.4; 134.4; 131.8; 128.4; 128.3; 52.4; 21.1; 19.0 ppm.

Elemental analysis [calculated on the basis of the Formula C₉H₁₀Cl₂N₂O₂S (281.16)]:

Calculated: C, 38.45; H, 3.59; Cl 25.22; N 9.96; S 11.40%

Measured: C, 38.65; H, 3.45; Cl 25.20; N 9.90; S 11.18%

### Example 7

### 8-chloro-4-methyl-3,4-dihydrobenzo[1,2,3]thiadiazine-1,1-dioxide

The title compound is prepared according to the procedure of Example 1 using 8-chloro-4-methylbenzo[1,2,3]thiadiazine-1,1-dioxide (5.0 g; 0.022 mol) and platinum(IV)-oxide (0.5 g).

Yield, 4.9 g, white crystals (96%)

Melting point, 202-203° C. (acetone)

IR (KBr): 3328 (NH); 1336, 1168 (S═O) cm⁻¹.

¹HNMR (DMSO, 400 MHz): 8.64 (1H, s); 7.57-7.50 (2H, m); 7.42 (1H, d, J=7.4 Hz); 6.00 (1H, s); 4.25 (1H, m); 1.34 (3H, d, J=6.7 Hz) ppm.

¹³CNMR (DMSO, 400 MHz): 144.9; 134.9; 132.6; 130.0; 129.4; 126.2; 52.5; 19.2 ppm.

Elemental analysis [calculated on the basis of the Formula C₈H₉ClN₂O₂S (232.69)]:

Calculated: C, 41.29; H, 3.90; Cl 15.24; N 12.04; S 13.78%

Measured: C, 41.90; H, 3.98; Cl 15.06; N 12.12; S 13.52%

### Example 8

### 4-Methyl-3,4-dihydrobenzo[1,2,3]thiadiazine-1,1-dioxide

The title compound is prepared according to the procedure of Example 1 using 4-methylbenzo[1,2,3]thiadiazine-1,1-dioxide (3.34 g; 0.017 mol) and platinum(IV)-oxide (0.3 g).

Yield, 3.3 g, white crystals (98%)

Melting point, 150-152° C. (2-propanol)

IR (KBr): 3179 (NH); 1300, 1172 (S═O) cm⁻¹.

¹HNMR (DMSO, 400 MHz): 8.51 (1H, d, J=2.4 Hz); 7.72 (1H, dd, J=1.3; 7.8 Hz); 7.56 (1H, dt, J=1.4; 7.6 Hz); 7.47-7.40 (2H, m); 5.91 (1H, t, J=3.0 Hz); 4.25-4.21 (1H, m); 1.35 (3H, d, J=6.8 Hz) ppm.

¹³CNMR (DMSO, 400 MHz): 141.5; 136.8; 131.9; 127.5; 126.9; 123.7; 51.6; 19.1 ppm.

Elemental analysis [calculated on the basis of the Formula C₈H₁₀N₂O₂S (198.25)]:

Calculated: C, 48.47; H, 5.08; N, 14.13; S 16.17%

Measured: C, 48.51; H, 5.12; N, 13.91; S 15.86%

### Example 9

### 8-chloro-4-methyl-7-methoxy-3,4-dihydrobenzo[1,2,3]thiadiazine-1,1-dioxide

The title compound is produced according to the procedure of Example 1 using 8-chloro-4-methyl-7-methoxybenzo[1,2,3]thiadiazine-1,1-dioxide (3.0 g; 0.0115 mol) and platinum(IV)-oxide (0.2 g).

Yield, 2.7 g, white crystals (90%)

Melting point, 231-233° C. (acetonitrile)

IR (KBr): 3277; 3189 (NH); 1288, 1164 (S═O) cm⁻¹.

¹HNMR (DMSO, 400 MHz): 8.57 (1H, s); 7.38 (1H, d, J=8.9 Hz); 7.34 (1H, d, J=8.9 Hz); 5.93 (1H, s); 4.22-4.19 (1H, m); 3.90 (3H, s); 1.31 (3H, d, J=6.7 Hz) ppm.

¹³CNMR (DMSO, 400 MHz): 153.8; 135.8; 135.5; 126.9; 118.0; 115.7; 56.8; 52.1; 19.2 ppm.

Elemental analysis [calculated on the basis of the Formula C₉H₁₁ClN₂O₃S (262.72)]:

Calculated: C, 41.15; H, 4.22; Cl 13.49; N 10.66; S 12.20%

Measured: C, 41.37; H, 4.17; Cl 13.33; N 10.84; S 12.08%

### Example 10

### 7,8-Dimethoxy-4-methyl-3,4-dihydrobenzo[1,2,3]thiadiazine-1,1-dioxide

The title compound is prepared according to the procedure of Example 1 using 7,8-dimethoxy-4-methylbenzo[1,2,3]thiadiazine-1,1-dioxide (7.2 g; 0.028 mol) and platinum(IV)-oxide (0.7 g).

Yield, 6.6 g, white crystals (91%)

Melting point: 211-213° C. (methanol)

IR (KBr): 3013 (NH); 1333, 1188 (S═O) cm⁻¹.

¹HNMR (DMSO, 200 MHz): 8.28 (1H, s); 7.26 (1H, d, J=8.9 Hz); 7.09 (1H, dd, J=0.8; 9.5 Hz); 5.77 (1H, d, J=1.2 Hz); 4.19-4.06 (1H, m); 3.85 (3H, s); 3.82 (3H, s); 1.29 (3H, d, J=6.7 Hz) ppm.

Elemental analysis [calculated on the basis of the Formula C₁₀H₁₄N₂O₄S (258.30)]:

Calculated: C, 46.50; H, 5.46; N, 10.85; S 12.41%

Measured: C, 46.67; H, 5.53; N, 10.86; S 12.20%

### Example 11

### 4-Methyl-8-methoxy-3,4-dihydrobenzo[1,2,3]thiadiazine-1,1-dioxide

The title compound is prepared according to the procedure of Example 1 using 4-methyl-8-methoxybenzo[1,2,3]thiadiazine-1,1-dioxide (2.26 g; 0.01 mol) and platinum(IV)-oxide (0.4 g).

Yield, 1.94 g, white crystals (85%)

Melting point, 231-233° C. (ethanol)

IR (KBr): 3276, 3185 (NH); 1277, 1125 (S═O) cm⁻¹.

¹HNMR (DMSO, 200 MHz): 8.28 (1H, s); 7.48 (1H, t, J=8.1 Hz); 7.06 (1H, d, J=8.3 Hz); 6.95 (1H, d, J=7.8 Hz); 5.81 (1H, d, J=3.0 Hz); 4.16 (1H, dq, J=2.7; 6.6 Hz); 3.86 (3H, s); 1.31 (3H, d, J=6.8 Hz) ppm.

¹³CNMR (DMSO, 200 MHz): 156.6; 143.8; 132.8; 125.6; 118.6; 110.8; 56.5; 52.0; 19.2 ppm.

Elemental analysis [calculated on the basis of the Formula C₉H₁₂N₂O₃S (228.27)]:

Calculated: C, 47.36; H, 5.30; N, 12.27; S 14.05%

Measured: C, 47.30; H, 5.31; N, 12.26; S 13.98%

### Example 12

### 7,8-Dichloro-3,4-dihydrobenzo[1,2,3]thiadiazine-1,1-dioxide

The title compound is prepared according to the procedure of Example 1 using 7,8-dichlorobenzo[1,2,3]thiadiazine-1,1-dioxide (2.0 g; 0.008 mol) and platinum(IV)-oxide (0.2 g).

Yield, 2.0 g, white crystals (99%)

Melting point, 215-217° C. (methanol)

IR (KBr): 3345, 3188 (NH); 1321, 1179 (S═O) cm⁻¹.

¹HNMR (DMSO, 200 MHz): 8.65 (1H, d, J=3.0 Hz); 7.82 (1H, d, J=8.5 Hz); 7.39 (1H, d, J=8.5 Hz); 6.18 (1H, q, J=3.0 Hz); 4.10 (2H, d, J=3.0 Hz) ppm.

¹³CNMR (DMSO, 200 MHz): 139.3; 137.1; 132.7; 131.6; 128.3; 127.7; 48.2 ppm.

Elemental analysis [calculated on the basis of the Formula C₇H₆Cl₂N₂O₂S (253.11)]:

Calculated: C 33.22; H 2.39; 0128.01; N 11.07; S 12.67%

Measured: C, 33.65; H, 2.43; Cl 27.84; N 10.94; S 12.41%

### Example 13

### 6-Methoxy-3,4-dihydrobenzo[1,2,3]thiadiazine-1,1-dioxide

The title compound is prepared according to the procedure of Example 1 using 6-methoxybenzo[1,2,3]thiadiazine-1,1-dioxide (1.49 g; 0.007 mol) and platinum(IV)-oxide (0.1 g).

Yield, 1.49 g, white crystals (99%)

Melting point, 181-182° C. (ethanol)

IR (KBr): 3350, 3161 (NH); 1289, 1173 (S═O) cm⁻¹.

¹HNMR (DMSO, 400 MHz): 8.29 (1H, d, J=3.5 Hz); 7.66 (1H, d, J=8.7 Hz); 6.98 (1H, dd, J=2.6; 8.8 Hz); 6.88 (1H, d, J=2.5 Hz); 5.98 (1H, q, J=3.9 Hz); 4.03 (2H, d, J=3.8 Hz); 3.80 (3H, s) ppm.

¹³CNMR (DMSO, 400 MHz): 161.3; 139.6; 129.6; 125.6; 114.0; 111.1; 55.7; 47.8 ppm.

Elemental analysis [calculated on the basis of the Formula C₈H₁₀N₂O₃S (214.24)]:

Calculated: C, 44.85; H, 4.70; N, 13.08; S 14.97%

Measured: C, 44.81; H, 4.75; N, 13.73; S 14.73%

### Example 14

### 3,4-Dihydrobenzo[1,2,3]thiadiazine-1,1-dioxide

The title compound is prepared according to the procedure of Example 1 using benzo[1,2,3]thiadiazine-1,1-dioxide (for the preparation of the starting compound, see: J. F. King, B. L. Huston, A. Hawson, D. M. Deaken, D. R. K. Harding, Can. J. Chem., 1971, 49, 936-942) (2.73 g; 0.015 mol) and platinum(IV)-oxide (0.6 g).

Yield, 2.0 g, white crystals (72%)

Melting point, 134-136° C. (hexane-ethylacetate 1:1)

IR (KBr): 3335, 3169 (NH); 1300, 1173 (S═O) cm⁻¹.

¹HNMR (CDCl₃, 400 MHz): 7.86 (1H, dd, J=1.4; 7.8 Hz); 7.50 (1H, dt, J=1.4; 7.6 Hz); 7.43 (1H, t, J=7.5 Hz); 7.18 (1H, dd, J=0.4, 7.8 Hz); 5.74 (1H, s); 4.97 (1H, s); 4.24 (2H, s) ppm.

¹³CNMR (CDCl₃, 400 MHz): 190.5; 135.7; 132.2; 128.0; 126.4; 124.0; 48.6 ppm.

Elemental analysis [calculated on the basis of the Formula C₇H₈N₂O₂S (184.22)]:

Calculated: C, 45.64; H, 4.38; N, 15.21; S 17.41%

Measured: C, 45.71; H, 4.40; N, 14.68; S 17.32%

### Example 15

### 8-chloro-7-methoxy-3,4-dihydrobenzo[1,2,3]thiadiazine-1,1-dioxide

The title compound is prepared according to the procedure of Example 1 using 8-chloro-7-methoxybenzo[1,2,3]thiadiazine-1,1-dioxide (1.23 g; 0.005 mol) and platinum(IV)-oxide (0.1 g).

Yield, 1.04 g, white crystals (84%)

Melting point, 209-210° C. (acetonitrile)

IR (KBr): 3277; 3189 (NH); 1288, 1164 (S═O) cm⁻¹.

¹HNMR (DMSO, 400 MHz): 8.45 (1H, d, J=1.8 Hz); 7.35 (1H, d, J=8.8 Hz); 7.29 (1H, d, J=8.8 Hz); 6.04 (1H, d, J=3.0 Hz); 4.02 (2H, d, J=3.0 Hz); 3.89 (3H, s) ppm.

Elemental analysis [calculated on the basis of the Formula C₈H₉ClN₂O₃S (248.70)]:

Calculated: C, 38.64; H, 3.65; Cl 14.26; N 11.26; S 12.89%

Measured: C, 39.65; H, 3.72; Cl 14.27; N 10.97; S 12.97%

### Example 16

### 7,8-Dichloro-2,4-dimethyl-3,4-dihydrobenzo[1,2,3]thiadiazine-1,1-dioxide

The title compound is prepared according to the procedure of Example 1 using 7,8-dichloro-2,4-dimethylbenzo[1,2,3]thiadiazine-1,1-dioxide (3.77 g; 0.0135 mol) and platinum(IV)-oxide (0.2 g).

Yield, 3.1 g, white crystals (82%)

Melting point, 134-135° C. (methanol)

IR (KBr): 3262 (NH); 1311, 1145 (S═O) cm⁻¹.

¹HNMR (CDCl₃, 400 MHz): 7.57 (1H, d, J=8.5 Hz); 7.09 (1H, d, J=8.5 Hz); 4.85 (1H, d, J=5.9 Hz); 4.36 (1H, kv, J=6.8 Hz); 3.02 (3H, s); 1.49 (3H, d, J=6.9 Hz) ppm.

¹³CNMR (CDCl₃, 400 MHz): 142.6; 135.0; 133.5; 133.1; 129.7; 125.9; 51.0; 35.5; 20.2 ppm.

Elemental analysis [calculated on the basis of the Formula C₉H₁₀Cl₂N₂O₂S (281.16)]:

Calculated: C, 38.45; H, 3.59; Cl 25.22; N 9.96; S 11.40%

Measured: C, 38.90; H, 3.63; Cl 25.27; N 9.93; S 11.20%

### Example 17

### 7,8-Dichloro-4-ethyl-2-methyl-3,4-dihydrobenzo[1,2,3]thiadiazine-1,1-dioxide

The title compound is prepared according to the procedure of Example 1 using 7,8-dichloro-4-ethyl-2-methylbenzo[1,2,3]thiadiazine-1,1-dioxide (1.0 g; 0.0034 mol) and platinum(IV)-oxide (0.1 g).

Yield, 0.95 g, white crystals (94%)

Melting point, 139-141° C. (ethanol)

IR (KBr): 1311, 1142 (S═O) cm⁻¹.

¹HNMR (CDCl₃, 200 MHz): 7.56 (1H, d, J=8.4 Hz); 7.17 (1H, dd, J=0.7; 8.4 Hz); 4.84 (1H, d, J=5.9 Hz); 4.04 (1H, m); 3.01 (3H, s); 2.10-1.74 (2H, m); 1.05 (3H, t, J=7.5 Hz) ppm.

¹³CNMR (CDCl₃, 200 MHz): 142.1; 135.5; 133.6; 133.0; 129.8; 125.9; 57.3; 35.4; 27.5; 10.5 ppm.

Elemental analysis [calculated on the basis of the Formula C₁₀H₁₂Cl₂N₂O₂S (295.19)]:

Calculated: C, 40.69; H, 4.10; Cl 24.02; N 9.49; S 10.86%

Measured: C, 40.45; H, 4.20; Cl 23.91; N 9.29; S 10.72%

### Example 18

### 7,8-Dichloro-2,4,5-trimethyl-3,4-dihydrobenzo[1,2,3]thiadiazine-1,1-dioxide

The title compound is produced according to the procedure of Example 1 with the difference that 7,8-dichloro-2,4,5-trimethyl-benzo[1,2,3]thiadiazine-1,1-dioxide (2.20 g; 0.0075 mol) and platinum(IV)-oxide (0.15 g) are used.

Yield, 1.4 g, white crystals (63%)

Melting point, 101-102° C. (2-propanol)

IR (KBr): 3268, 3242 (NH); 1309, 1130 (S═O) cm⁻¹.

¹HNMR (CDCl₃, 400 MHz): 7.40 (1H, q, J=0.6 Hz); 5.04 (1H, s); 4.17-4.13 (1H, m); 2.99 (3H, s); 2.29 (3H, d, J=0.6 Hz); 1.52 (3H, d, J=6.7 Hz) ppm.

¹³CNMR (CDCl₃, 400 MHz): 141.7; 135.1; 135.0; 134.8; 133.2; 127.2; 51.4; 35.3; 19.0; 18.2 ppm.

Elemental analysis [calculated on the basis of the Formula C₁₀H₁₂Cl₂N₂O₂S (295.19)]:

Calculated: C, 40.69; H, 4.10; Cl 24.02; N 9.49; S 10.86%

Measured: C, 40.51; H, 4.07; Cl 23.80; N 9.44; S 10.62%

### Example 19

### 2,4-Dimethyl-8-chloro-7-methoxy-3,4-dihydrobenzo[1,2,3]thiadiazine-1,1-dioxide

The title compound is prepared according to the procedure of Example 1 using 2,4-dimethyl-8-chloro-7-methoxybenzo[1,2,3]thiadiazine-1,1-dioxide (2.13 g; 0.00775 mol) and platinum(IV)-oxide (0.2 g).

Yield, 1.87 g, white crystals (86%)

Melting point, 141-143° C. (ethanol)

IR (KBr): 3312 (NH); 1337, 1176 (S═O) cm⁻¹.

¹HNMR(CDCl₃, 400 MHz): 7.11 (1H, dq, J=0.6; 8.7 Hz); 7.07 (1H, d, J=8.8 Hz); 4.8 (1H, s); 4.33 (1H, q, J=6.6 Hz); 3.92 (3H, s); 3.01 (3H, s); 1.46 (3H, d, J=6.9 Hz) ppm.

¹³CNMR (CDCl₃, 400 MHz): 154.6; 135.1; 134.1; 125.8; 119.9; 115.2; 56.6; 50.5; 35.3; 20.4 ppm.

Elemental analysis [calculated on the basis of the Formula C₁₀H₁₃ClN₂O₃S (276.74)]:

Calculated: C, 43.40; H, 4.73; Cl 12.81; N 10.12; S 11.59%

Measured: C, 43.62; H, 4.90; Cl 13.30; N 10.12; S 11.74%

### Example 20

### 7,8-Dichloro-2-methyl-3,4-dihydrobenzo[1,2,3]thiadiazine-1,1-dioxide

The title compound is prepared according to the procedure of Example 1 starting from 7,8-dichloro-2-methylbenzo[1,2,3]thiadiazine-1,1-dioxide (3.98 g; 0.015 mól) and platinum(IV)-oxide (0.5 g).

Yield, 1.33 g, white crystals (33%)

Melting point, 124-125° C. (ethanol)

IR (KBr): 3241 (NH); 1318, 1143 (S═O) cm⁻¹

¹HNMR (DMSO, 200 MHz): 7.84 (1H, d, J=8.6 Hz); 7.40 (1H, d, J=8.6 Hz); 6.36 (1H, t, J=4.3 Hz); 4.17 (2H, d, J=4.0 Hz); 2.94 (3H, s) ppm.

¹³CNMR (DMSO, 200 MHz): 138.8; 134.7; 133.2; 131.9; 128.7; 127.8; 41.5; 35.2 ppm.

Elemental analysis [calculated on the basis of the Formula a C₈H₈Cl₂N₂O₂S (267.14)]:

Calculated: C, 35.97; H, 3.02; Cl 26.54; N 10.49; S 12.00%

Measured: C, 36.60; H, 2.99; Cl 26.28; N 10.35; S 12.09%

### Example 21

### 7,8-Dichloro-2-ethyl-4-methyl-3,4-dihydrobenzo[1,2,3]thiadiazine-1,1-dioxide

7,8-Dichloro-4-methyl-3,4-dihydrobenzo[1,2,3]thiadiazine-1,1-dioxide (the compound of Example 3; 1.0 g; 0.0037 mol) is dissolved in N,N-dimethylformamide (6 ml) and to this solution potassium-tent-butylate (0.83 g; 0.0074 mol) is added at the temperature of 10° C. and subsequently ethyl iodide (0.6 ml; 1.15 g; 0.0074 mol) are added dropwise to the reaction mixture. The mixture is stirred at 20° C. for one hour, poured onto an ice-water mixture, the precipitated crystals are filtered and washed with water.

Yield, 0.82 g, white crystals (75%)

Melting point, 135-136° C. (hexane-ethyl acetate 1:1).

IR (KBr): 3433 (NH); 1314, 1141 (S═O) cm⁻¹.

¹HNMR (CDCl₃, 400 MHz): 7.55 (1H, d, J=8.4 Hz); 7.05 (1H, d, J=8.5 Hz); 4.73 (1H, s); 4.27 (1H, kv, J=6.7 Hz); 3.53-3.44 (1H, m); 3.28-3.19 (1H, m); 1.50 (3H, d, J=6.9 Hz); 1.30 (3H, t, J=7.1 Hz) ppm.

¹³CNMR (CDCl₃, 400 MHz): 143.0; 135.9; 133.6; 132.9; 129.7; 125.9; 52.3; 42.1; 20.5; 12.3 ppm.

Elemental analysis [calculated on the basis of the Formula C₁₀H₁₂Cl₂N₂O₂S (295.19)]:

Calculated: C, 40.69; H, 4.10; Cl 24.02; N 9.49; S 10.86%

Measured: C, 40.14; H, 4.25; Cl 123.62; N 9.44; S 10.81%

### Example 22

### 2,4-Diethyl-7,8-dichloro-3,4-dihydrobenzo[1,2,3]thiadiazine-1,1-dioxide

The title compound is prepared according to the procedure described in Example 22 using 7,8-dichloro-4-ethyl-3,4-dihydro-benzo[1,2,3]thiadiazine-1,1-dioxide (compound of Example 4; 2.8 g; 0.01 mol), potassium tert-butylate (2.0 g; 0.018 mol) and ethyl iodide (1.5 ml; 3.0 g; 0.019 mol).

Yield, 2.6 g, white crystals (71%)

Melting point, 118-120° C. (2-propanol).

IR (KBr): 1315, 1163 (S═O) cm⁻¹.

¹HNMR (CDCl₃, 400 MHz): 7.55 (1H, d, J=8.5 Hz); 7.06 (1H, d, J=8.4 Hz); 4.73 (1H, s); 3.96 (1H, s); 3.51-3.45 (1H, m); 3.30-3.22 (1H, m); 2.10-1.90 (1H, m); 1.88-1.75 (1H, m); 1.31 (3H, t, J=7.1 Hz); 1.08 (3H, t,) ppm.

¹³CNMR (CDCl₃, 400 MHz): 142.3; 136.3; 133.6; 132.8; 129.6; 126.0; 58.1; 42.4; 27.5; 12.4; 10.7 ppm.

Elemental analysis [calculated on the basis of the Formula C₁₁H₁₄Cl₂N₂O₂S (309.22)]:

Calculated: C, 42.73; H, 4.56; Cl 22.93; N 9.06; S 10.37%

Measured: C, 43.13; H, 4.73; Cl 23.06; N 9.09; S 10.42%

### Example 23

### 2,4-Dimethyl-8-chloro-3,4-dihydrobenzo[1,2,3]thiadiazine-1,1-dioxide

The title compound is produced according to the procedure disclosed in Example 21 using 8-chloro-4-methyl-3,4-dihydro-benzo[1,2,3]thiadiazine-1,1-dioxide (compound of Example 7, 1.5 g; 0.0065 mol), potassium tent-butylate (1.5 g; 0.013 mol) and methyl iodide (0.8 ml; 1.85 g; 0.013 mol).

Yield, 1.3 g, white crystals (81%)

Melting point, 128-130° C. (2-propanol).

IR (KBr): 3263 (NH); 1307, 1141 (S═O) cm⁻¹.

¹HNMR (CDCl₃, 200 MHz): 7.36-7.43 (2H, m); 7.08-7.12 (1H, m); 4.80 (1H, d, J=7.0 Hz); 4.35 (1H, kv, J=7.0 Hz); 3.02 (3H, s); 1.51 (3H, d, J=7.0 Hz) ppm.

Elemental analysis [calculated on the basis of the Formula C₉H₁₁ClN₂O₂S (246.72)]:

Calculated: C, 43.82; H, 4.49; Cl 14.37; N 11.35; S 13.00%

Measured: C, 44.28; H, 4.30; Cl 14.19; N 11.35; S 12.83%

### Example 24

### 2-Ethyl-8-chloro-4-methyl-3,4-dihydrobenzo[1,2,3]thiadiazine-1,1-dioxide

The title compound is prepared according to the procedure described in Example 21 using 8-chloro-4-methyl-3,4-dihydrobenzo[1,2,3]thiadiazine-1,1-dioxide (compound of Example 7; 1.0 g; 0.0043 mol), potassium-tent-butylate (0.83 g; 0.074 mol) and ethyl iodide (0.6 ml; 1.15 g; 0.0074 mol).

Yield, 0.7 g, white crystals (63%).

Melting point, 79-80° C. (hexane-ethylacetate 1:1).

IR (KBr): 1314, 1138 (S═O) cm

¹HNMR (CDCl₃, 400 MHz): 7.39-7.06 (2H, m); 7.11-7.06 (1H, m); 4.72 (1H, d, J=6.2 Hz); 4.28 (1H, kv, J=6.8 Hz); 3.53-3.42 (1H, m); 3.40-3.17 (1H, m); 1.52 (3H, d, J=7.0 Hz); 1.30 (3H, t, J=7.2 Hz) ppm.

¹³CNMR (CDCl₃, 400 MHz): 144.9; 134.2; 132.2; 131.2; 130.0; 125.1; 52.7; 41.9; 20.6; 12.2 ppm.

Elemental analysis [calculated on the basis of the Formula C₁₀H₁₃ClN₂O₂S (260.74)]:

Calculated: C, 46.06; H, 5.03; Cl 13.60; N 10.74; S 12.30%

Measured: C, 45.87; H, 4.93; Cl 13.38; N 10.79; S 12.21%

### Example 25

### 7,8-Dichloro-2,4,6-trimethyl-3,4-dihydrobenzo[1,2,3]thiadiazine-1,1-dioxide

The title compound is prepared according to the procedure disclosed in Example 21 using 7,8-dichloro-4,6-dimethyl-3,4-dihydrobenzo[1,2,3]thiadiazine-1,1-dioxide (compound of Example 6; 0.7 g; 0.0025 mol), potassium-tert-butylate (0.5 g; 0.0045 mol) and methyl iodide (0.3 ml; 0.71 g; 0.005 mol).

Yield, 0.43 g, white crystals (58%)

Melting point, 141-142° C. (ethanol).

IR (KBr): 3318 (NH); 1333, 1163 (S═O) cm⁻¹.

¹HNMR (CDCl₃, 400 MHz): 7.52 (1H, s); 6.13 (1H, d, J=3.9 Hz); 4.41 (1H, m); 2.91 (3H, s); 2.44 (3H, s); 1.33 (3H, d, J=6.6 Hz) ppm.

Elemental analysis [calculated on the basis of the Formula C₁₀H₁₂Cl₂N₂O₂S (295.19)]:

Calculated: C, 40.69; H, 4.10; Cl 24.02; N 9.49; S 10.86%

Measured: C, 40.49; H, 4.29; Cl 24.08; N 9.14; S 10.74%

### Example 26

### 7,8-Dichloro-3,4-dimethyl-3,4-dihydrobenzo[1,2,3]thiadiazine-1,1-dioxide

The mixture of 7,8-dichloro-4-methyl-3,4-dihydro-benzo[1,2,3]thiadiazine-1,1-dioxide (compound of Example 3; 4.8 g; 0.018 mol), formaldehyde (4.0 g; 0.13 mol) is hydrogenated in the mixture of tetrahydrofurane (150 ml) and acetic acid (4 ml) at 10 bar hydrogen pressure in the presence of palladium-charcoal catalyst (10% by weight; 0.5 g). After the calculated amount of hydrogen is used up, the catalyst is filtered off and the filtrate is evaporated.

Yield, 4.5 g, white crystals (89%)

Melting point, 194-195° C. (ethanol).

IR (KBr): 1329, 1160 (S═O) cm⁻¹.

¹HNMR (CDCl₃, 200 MHz): 7.55 (1H, d, J=8.4 Hz); 7.10 (1H, dd, J=0.7; 8.4 Hz); 5.87 (1H, s); 4.05 (1H, q, J=6.6 Hz); 2.79 (3H, s); 1.44 (3H, d, J=6.6 Hz) ppm.

¹³CNMR (CDCl₃, 200 MHz): 141.4; 136.0; 133.6; 132.8; 130.4; 126.3; 58.5; 43.0; 12.9 ppm.

Elemental analysis [calculated on the basis of the Formula C₉H₁₀Cl₂N₂O₂S (281.16)]:

Calculated: C, 38.45; H, 3:59; Cl 25.22; N 9.96; S 11.40%

Measured: C, 38.50; H, 3.63; Cl 25.01; N 9.72; S 11.02%

### Example 27

### 7,8-Dichloro-3-ethyl-4-methyl-3,4-dihydrobenzo[1,2,3]thiadiazine-1,1-dioxide

The title compound is produced according to the procedure described in Example 26 with the modification that 7,8-dichloro-4-methyl-3,4-dihydrobenzo[1,2,3]thiadiazine-1,1-dioxide (compound of Example 3; 3.0 g; 0.011 mol), acetaldehyde (2.0 ml; 1.6 g; 0.036 mol) and palladium-charcoal catalyst (10% by weight; 0.3 g) are used.

Yield, 2.8 g, white crystals (86%)

Melting point, 172-174° C. (hexane:ethylacetate 1:1).

IR (KBr): 3191 (NH); 1341, 1183 (S═O) cm⁻¹.

¹HNMR. (CDCl₃, 400 MHz): 7.54 (1H, d, J=8.4 Hz); 7.09 (1H, dd, J=0.4; 8.4 Hz); 5.77 (1H, s); 4.17 (1H, q, J=6.9 Hz); 3.00-2.90 (1H, m); 2.83-2.74 (1H, m); 1.42 (3H, d, J=6.9 Hz); 1.25 (3H, t, J=7.1 Hz) ppm.

¹³CNMR (CDCl₃, 400 MHz): 142.8; 137.3; 134.6; 133.8; 131.6; 127.5; 59.3; 50.1; 13.6; 12.9 ppm.

Elemental analysis [calculated on the basis of the Formula C₁₀H₁₂Cl₂N₂O₂S (295.19)]:

Calculated: C, 40.69; H, 4.10; Cl 24.02; N 9.49; S 10.86%

Measured: C, 40.76; H, 4.06; Cl 23.55; N 9.44; S 10.68%

### Example 28

### 7,8-Dichloro-4-methyl-3-propyl-3,4-dihydrobenzo[1,2,3]thiadiazine-1,1-dioxide

The title compound is produced according to the procedure described in Example 26 with the difference that 7,8-dichloro-4-methyl-3,4-dihydrobenzo[1,2,3]thiadiazine-1,1-dioxide (compound of the Formula 3; 3.0 g; 0.011 mol), propionaldehyde (3.0 ml; 2.4 g; 0.042 mol) and palladium-charcoal catalyst (10% by weight; 0.3 g) are used.

Yield, 2.0 g, white crystals (59%)

Melting point, 164-165° C. (hexane:ethylacetate 1:1)

IR (KBr) 3193 (NH); 1333, 1180 (5=0) cm⁻¹.

¹HNMR (CDCl₃, 200 MHz): 7.54 (1H, d, J=8.4 Hz); 7.09 (1H, dd, J=0.6; 8.4 Hz); 5.81 (1H, s); 4.13 (1H, q, J=6.9 Hz); 2.91-2.83 (1H, m); 2.66-2.58 (1H, m); 1.74-1.64 (2H, m); 1.42 (3H, d, J=7.0 Hz); 0.97 (3H, t, J=7.4 Hz) ppm

¹³CNMR (CDCl₃, 200 MHz): 141.8; 135.2; 133.5; 132.7; 130.4, 126.4; 56.4; 53.6; 19.8; 12.5; 11.3 ppm.

Elemental analysis [calculated on the basis of the Formula C₁₁H₁₄Cl₂N₂O₂S (309.22)]:

Calculated: C, 42.73; H, 4.56; Cl 22.93; N 9.06; S 10.37%

Measured: C, 43.07; H, 4.33; Cl 22.76; N 9.01; S 10.16%

### Example 29

### 3,4-Diethyl-7-chloro-3,4-dihydrobenzo[1,2,3]thiadiazine-1,1-dioxide

The title compound is produced according to the procedure of Example with the modification that 4-ethyl-7-chloro-3,4-dihydrobenzo[1,2,3]thiadiazine-1,1-dioxide (compound of the Formula 2; 1.5 g; 0.0061 mol), acetaldehyde (1.0 ml; 0.8 g; 0.018 mol) and palladium-charcoal catalyst (10 percent by weight, 0.2 g) are used.

Yield, 1.6 g, white crystals (96%).

Melting point, 109-111° C. (hexane-ethyl acetate 1:1)

IR (KBr): 3201 (NH); 1351, 1174 (S═O) cm⁻¹.

¹HNMR (CDCl₃, 400 MHz): 7.79 (1H, d, J=2.1 Hz); 7.43 (1H, dd, J=2.2; 8.4 Hz); 7.17 (1H, d, J=8.4 Hz); 5.80 (1H, s); 3.80 (1H, t, J=6.1 Hz); 3.10-3.00 (2H, m); 2.05-1.93 (1H, m); 1.83-1.60 (1H, m); 1.24 (3H, t, J=7.1 Hz); 1.03 (3H, s) ppm.

¹³CNMR (CDCl₃, 400 MHz): 137.5; 137.3; 133.9; 131.9; 129.0; 124.6; 62.8; 49.5; 23.5; 12.1; 10.7 ppm.

Elemental analysis [calculated on the basis of the Formula C₁₁H₁₅ClN₂O₂S (274.77)]:

Calculated: C, 48.08; H, 5.50; Cl 12.90; N 10.20; S 11.67%

Measured: C, 48.47; H, 5.53; Cl 12.86; N 10.15; S 11.53%

### Example 30

### 4-Ethyl-3-isopropyl-7-chloro-3,4-dihydrobenzo[1,2,3]thia diazine-1,1-dioxide

The title compound is produced according to the procedure described in Example 26 using 4-ethyl-7-chloro-3,4-dihydrobenzo[1,2,3]thiadiazine-1,1-dioxide (compound of Example 2; 7.8 g; 0.032 mol), acetone (10 ml; 7.9 g; 0.136 mol) and palladium-charcoal catalyst (10% by weight, 0.8 g).

Yield, 7.3 g, white crystals (80%)

Melting point, 99-101° C. (hexane-ethylacetate 1:1)

IR (KBr): 3169 (NH); 1313, 1176 (S═O) cm⁻¹.

¹HNMR (CDCl₃, 400 MHz): 7.83 (1H, d, J=2.2 Hz); 7.45 (1H, d, J=2.2; 8.4 Hz); 7.19 (1H, d, J=8.4 Hz); 5.49 (1H, s); 4.05 (1H, t, J=5.5 Hz); 3.41-3.27 (1H, m); 2.07-1.77 (2H, m); 1.26 (3H, d, J=6.2 Hz); 1.15 (3H, d, J=6.6 Hz); 0.95 (3H, t, J=7.5 Hz) ppm.

¹³CNMR (CDCl₃, 400 MHz): 138.5; 137.6; 134.1; 132.1; 128.9; 125.1; 60.3; 51.9; 23.3; 21.0; 18.1; 9.9 ppm.

Elemental analysis [calculated on the basis or Formula C₁₂H₁₇ClN₂O₂S (288.80)]:

Calculated: C, 49.91; H, 5.93; Cl 12.28; N 9.70; S 11.10%

Measured: C, 50.29; H, 5.99; Cl 12.00; N 9.65; S 10.97%

### Example 31

### 7,8-Dichloro-3,4,5-trimethyl-3,4-dihydrobenzo[1,2,3]thiadiazine-1,1-dioxide

The title compound is produced according to the procedure of Example starting from 7,8-dichloro-4,5-dimethyl-3,4-dihydrobenzo[1,2,3]thiadiazine-1,1-dioxide (compound of Example 5; 1.21 g; 0.0043 mol), formaldehyde (1.95 g; 0.0043 mol) and palladium-charcoal catalyst (10% by weight, 0.6 g).

Yield, 1.03 g, white crystals (81%)

Melting point, 236-238° C. (acetonitrile)

IR (KBr): 3182 (NH); 1323, 1178 (S═O) cm⁻¹.

¹HNMR (CDCl₃, 400 MHz): 8.82 (1H, s); 7.75 (1H, q, J=0.4 Hz); 4.21 (1H, q, J=6.7 Hz); 2.66 (3H, s); 2.33 (3H, s); 1.32 (3H, d, J=6.7 Hz) ppm.

¹³CNMR (CDCl₃, 400 MHz): 141.7; 136.6; 135.6; 134.7; 131.4; 125.8; 56.0; 42.5; 18.7; 10.0 ppm.

Elemental analysis [calculated on the basis of the Formula C₁₀H₁₂Cl₂N₂O₂S (295.19)]:

Calculated: C 40.69; H 4.10; Cl 24.02; N 9.49; S 10.86%

Measured: C 40.94; H 4.15; Cl 23.61; N 9.61; S 10.93%

### Example 32

### 3-Methyl-3,4-dihydrobenzo[1,2,3]thiadiazine-1,1-dioxide

### Step 1

### 3-Methylbenzo[1,2,3]thiadiazine-3-ium-1-oxide-1-oate

The solution of benzo[1,2,3]thiadiazine-1,1-dioxide (10.93 g, 0.06 mol) in tetrahydrofurane (90 ml) is added dropwise to the suspension of sodium hydride (50% by weight, 1.69 g; 0.066 mol) in tetrahydrofurane (60 ml) at room temperature (for the preparation of benzo[1,2,3]thiadiazine-1,1-dioxide, see: J. F. King, B. L. Huston, A. Hawson, D. M. Deaken, D. R. K. Harding, Can. J. Chem., 1971, 49, 936-942) (10.93 g; 0.06 mol). Subsequently methyl-iodide (11.2 ml; 25.55 g; 0.18 mol) is added dropwise and stirred for 30 minutes. After this period, the reaction mixture is evaporated and water is added to the residue and filtered.

Yield, 5.82 g, white crystals (49%)

Melting point, 228-230° C. (acetonitrile)

IR (KBr): 1285, 1172 (S═O) cm⁻¹.

¹HNMR (DMSO, 400 MHz): 8.90 (1H, q, J=0.8 Hz); 8.00-7.96 (1H, m); 7.88-7.84 (3H, m); 4.02 (3H, d, J=0.8 Hz) ppm.

¹³CNMR (CDCl₃, 400 MHz): 136.9; 135.5; 131.6; 126.8; 126.7; 126.1; 120.0; 51.9 ppm.

Elemental analysis [calculated on the basis of the Formula C₈H₈N₂O₂S (196.23)]:

Calculated: C, 48.97; H, 4.11; N, 14.28; S 16.34%

Measured: C, 49.07; H, 4.12; N, 14.08; S 16.24%

### Step 2

### 3-Methyl-3,4-dihydrobenzo[1,2,3]thiadiazine-1,1-dioxide

3-Methylbenzo[1,2,3]thiadiazine-3-ium-1-oxide-1-oate (product of step 1; 2.94 g; 0.015 mol) is added to acetic acid (90 ml) and the mixture is hydrogenated at room temperature in the presence of platinum(IV)-oxide (0.6 g) catalyst at 10 bar hydrogen pressure. The catalyst is filtered off and the filtrate is evaporated.

Yield, 2.22 g, white crystals (75%)

Melting point, 176-178° C. (ethanol)

IR (KBr): 3089 (NH); 1317, 1174 (S═O) cm⁻¹.

¹HNMR (CDCl₃, 200 MHz): 7.89-7.84 (1H, m); 7.55-7.40 (2H, m); 7.23-7.19 (1H, m); 5.11 (1H, s); 3.95 (2H, s); 2.85 (3H, s) ppm.

¹³CNMR (CDCl₃, 200 MHz): 136.3; 134.3; 132.1; 128.4; 126.7; 124.6; 58.5; 25.9 ppm.

Elemental analysis [calculated on the basis of the Formula C₈H₁₀N₂O₂S (198.25)]:

Calculated: C, 48.47; H, 5.08; N, 14.13; S 16.17%

Measured: C, 48.42; H, 5.11; N, 14.03; S 15.91%

### Example 33

### 3-Ethyl-3,4-dihydrobenzo[1,2,3]thiadiazine-1,1-dioxide

### Step 1

### 3-Ethylbenzo[1,2,3]thiadiazine-3-ium-1-oxide-1-oate

The title compound is produced according to the procedure described in Example 32, Step 1, starting from benzo[1,2,3]thiadiazine-1,1-dioxide (for the preparation of the starting compound, see: J. F. King, B. L. Huston, A. Hawson, D. M. Deaken, D. R. K. Harding, Can. J. Chem., 1971, 49, 936-942) (5.83 g; 0.032 mol), sodium hydride (50% by weight, 1.84 g; 0.0384 mol) and ethyl iodide (7.75 ml; 14.97 g; 0.096 mol).

Yield, 2.2 g, white crystals (32%)

Melting point, 137-138° C. (ethanol)

IR (KBr): 1286, 1164 (S═O) cm⁻¹.

¹HNMR (CDCl₃, 400 MHz): 8.14 (1H, s); 8.00 (1H, dd, J=0.5; 7.9 Hz); 7.89 (1H, dt, J=1.2; 7.6 Hz); 7.70 (1H, dt, J=1.2; 7.8 Hz); 7.62 (1H, d, J=7.5 Hz); 4.23 (2H, q, J=7.3 Hz); 1.68 (3H, t, J=7.3 Hz), ppm.

¹³CNMR (CDCl₃, 400 MHz): 136.2; 134.4; 132.2; 128.6; 127.2; 122.3; 121.7; 62.1; 15.4 ppm.

Elemental analysis [calculated on the basis of the Formula C₉H₁₀N₂O₂S (210.26)]:

Calculated: C, 51.41; H, 4.79; N, 13.32; S 15.25%

Measured: C, 51.68; H, 4.77; N, 13.19; S 15.21%

### Step 2

### 3-Ethyl-3,4-dihydrobenzo[1,2,3]thiadiazine-1,1-dioxide

3-Ethylbenzo[1,2,3]thiadiazine-3-ium-1-oxide-1-oate (product of step 1; 1.37 g; 0.0065 mol) is added to methanol (50 ml) and to this mixture, sodium borohydride (0.98 g, 0.026 mol) is added in small portions at 0-5° C. and the mixture is stirred for 3 hours at 50° C. The reaction mixture is evaporated, diluted with water and acidified with 10% hydrochloric acid solution. The crystals thus obtained are filtered.

Yield, 1.1 g, white crystals (80%)

Melting point, 146-147° C. (hexane-ethylacetate).

IR (KBr): 3132 (NH); 1350, 1174 (S═O) cm⁻¹.

¹HNMR (CDCl₃, 200 MHz): 7.85 (1H, dd, J=1.1; 7.7 Hz); 7.48 (1H, dt, J=1.5; 7.5 Hz); 7.43 (1H, dt, J=0.6; 7.6 Hz); 7.21 (1H, dt, J=0.6; 7.6 Hz); 4.98 (1H, s); 3.97 (2H, s); 2.96 (2H, q, J=7.1 Hz); 1.28 (3H, t, J=7.1 Hz) ppm.

¹³CNMR (CDCl₃, 200 MHz): 136.5; 134.6; 132.0; 128.3; 126.8; 124.6; 56.7; 52.9; 11.2 ppm.

Elemental analysis [calculated on the basis of the Formula C₉H₁₂N₂O₂S (212.27)]:

Calculated: C, 50.93; H, 5.70; N, 13.20; S 15.11%

Measured: C, 50.42; H, 5.84; N, 12.86; S 14.95%

### Example 34

### 3-Methyl-6-methoxy-3,4-dihydrobenzo[1,2,3]thiadiazine-1,1-dioxide

### Step 1

### 3-Methyl-6-methoxybenzo[1,2,3]thiadiazine-3-ium-1-oxide-1-oate

The title compound is produced according to the procedure described in Example 32, Step 1, starting from 6-methoxybenzo[1,2,3]thiadiazine-1,1-dioxide (2.12 g; 0.01 mol), sodium hydride (50% by weight; 0.53 g; 0.011 mol) and methyl iodide (1.87 ml; 4.26 g; 0.03 mol).

Yield, 1.0 g, white crystals (45%)

Melting point, 198-200° C. (ethanol)

IR (KBr): 1597 (C═N), 1257, 1128 (S═O) cm⁻¹.

¹HNMR (DMSO, 400 MHz): 8.74 (1H, s); 7.78 (1H, d, J=8.7 Hz) 7.52 (1H, dd, J=2.6; 8.7 Hz); 7.35 (1H, d, J=2.6 Hz); 4.00 (3H, d, J=0.6 Hz); 3.89 (3H, s); ppm.

¹³CNMR (DMSO, 400 MHz): 160.9; 135.8; 128.0; 122.9; 122.2; 121.8; 111.0; 56.1; 52.0 ppm.

Elemental analysis [calculated on the basis of the Formula C₉H₁₀N₂O₃S (226.26)]:

Calculated: C, 47.78; H, 4.46; N, 12.38; S 14.17%

Measured: C, 47.57; H, 4.46; N, 12.33; S 14.08%

### Step 2

### 3-Methyl-6-methoxy-3,4-dihydrobenzo[1,2,3]thiadiazine-1,1-dioxide

The title product is prepared according to the procedure of Example 33, Step 2 starting from 3-methyl-6-methoxybenzo[1,2,3]thiadiazine-3-ium-1-oxide-1-oate (product of Step 1, 0.90 g, 0.004 mol) and sidum borohydride (0.6 g; 0.016 mol).

Product, 0.73 g, white crystals (80%)

Melting point, 186-187° C. (ethanol)

IR (KBr): 3070 (NH); 1311, 1161 (S═O) cm⁻¹.

¹HNMR (CDCl₃, 400 MHz): 7.77 (1H, d, J=8.8 Hz); 6.93 (1H, dd, J=2.5; 8.8 Hz); 6.64 (1H, d, J=2.5 Hz); 5.12 (1H, s); 3.88 (2H, s); 3.84 (3H, s); 2.83 (3H, s) ppm.

¹³CNMR (CDCl₃, 400 MHz): 162.0; 136.6; 128.3; 126.3; 114.2; 111.3; 58.7; 55.6; 46.7 ppm.

Elemental analysis [calculated on the basis of the Formula C₉H₁₂N₂O₃S (228.27)]:

Calculated: C, 47.36; H, 5.30; N, 12.27; S 14.05%

Measured: C, 47.37; H, 5.32; N, 12.13; S 14.02%

### Example 35

### 3-Ethyl-6-methoxy-3,4-dihydrobenzo[1,2,3]thiadiazine-1,1-dioxide

### Step 1

### 3-Ethyl-6-methoxybenzo[1,2,3]thiadiazine-3-ium-1-oxide-1-oate

The title compound is produced according to the procedure described in Example 32, Step 1 starting from 6-methoxybenzo[1,2,3]thiadiazine-1,1-dioxide (7.00 g; 0.033 mol), sodium hydride (50% by weight; 1.90 g; 0.0396 mol) and ethyl iodide (13.3 ml; 25.74 g; 0.165 mol).

Yield, 1.6 g, white crystals (20%)

Melting point, 148-149° C. (ethanol)

IR (KBr): 1283, 1125 (S═O) cm⁻¹.

¹HNMR (CDCl₃, 400 MHz): 8.07 (1H, s); 7.89 (1H, d, J=8.8 Hz); 7.38 (1H, dd, J=2.5; 8.8 Hz); 7.00 (1H, d, J=2.4 Hz); 4.19 (2H, q, J=7.3 Hz); 3.89 (3H, s); 1.65 (3H, t, J=7.3 Hz) ppm.

¹³CNMR (CDCl₃, 400 MHz): 161.3; 132.7; 127.8; 123.1; 122.7; 122.0; 109.9; 61.0; 55.9; 14.3 ppm.

Elemental analysis [calculated on the basis of the Formula C₁₀H₁₂N₂O₃S (240.28)]:

Calculated: C, 49.99; H, 5.03; N, 11.66; S 13.34%

Measured: C, 50.03; H, 5.02; N, 11.64; S 13.29%

### Step 2

### 3-Ethyl-6-methoxy-3,4-dihydrobenzo[1,2,3]thiadiazine-1,1-dioxide

The title compound is prepared according to the procedure of Example 33, Step 2 starting from 3-ethyl-6-methoxybenzo[1,2,3]thiadiazine-3-ium-1-oxide-1-oate (product of reaction step 1; 1.08 g; 0.0045 mol) and sodium borohydride (0.68 g; 0.018 mol).

Yield, 0.95 g, white crystals (87%)

Melting point, 180-181° C. (ethanol)

IR (KBr): 3134 (NH); 1342, 1174 (S═O) cm⁻¹.

¹HNMR (CDCl₃, 400 MHz): 7.77 (1H, d, J=8.8 Hz); 6.92 (1H, dd, J=2.6; 8.8 Hz); 6.65 (1H, d, J=2.4 Hz); 4.94 (1H, s); 3.88 (2H, s); 3.83 (3H, s); 2.94 (2H, q, J=7.1 Hz); 1.27 (3H, t, J=7.1 Hz) ppm.

¹³CNMR (CDCl₃, 400 MHz): 162.0; 136.8; 128.7; 126.4; 114.1; 111.4; 57.7; 55.6; 52.8; 11.2 ppm.

Elemental analysis [calculated on the basis of the Formula C₁₀H₁₄N₂O₃S (242.30)]:

Calculated: C, 49.57; H, 5.82; N, 11.56; S 13.23%

Measured: C, 49.46; H, 5.80; N, 11.44; S 13.40%

### Example 36

### 7,8-Dichloro-3-methyl-3,4-dihydrobenzo[1,2,3]thiadiazine-1,1-dioxide

### Step 1

### 7,8 Dichloro-3-methylbenzo[1,2,3]thiadiazine-3-ium-1-oxide-1-oate

The title product is prepared according to the procedure disclosed in Example 32, Step 1, starting from 7,8-dichlorobenzo[1,2,3]thiadiazine-1,1-dioxide (1.26 g; 0.005 mol), sodium hydride (50% by weight; 0.26 g; 0.0055 mol) and methyl iodide (0.94 ml; 2.13 g; 0.015 mol).

Yield, 0.63 g, white crystals (48%)

Melting point, 240-241° C. (acetonitrile)

IR (KBr): 1290, 1143 (S═O) cm⁻¹.

¹HNMR (DMSO, 400 MHz): 8.92 (1H, s); 8.08 (1H, d, J=8.4 Hz); 7.86 (1H, d, J=8.4 Hz); 4.02 (3H, s) ppm.

¹³CNMR (DMSO, 400 MHz): 138.3; 136.6; 132.8; 129.7; 128.8; 127.1; 125.4; 51.8 ppm.

Elemental analysis [calculated on the basis of the Formula C₈H₆Cl₂N₂O₂S (265.12)]:

Calculated: C, 36.24; H, 2.28; Cl 26.74; N 10.57; S 12.09%

Measured: C, 36.59; H, 2.32; Cl 26.53; N 10.52; S 11.91%

### Step 2

### 7,8-Dichloro-3-methyl-3,4-dihydrobenzo[1,2,3]thiadiazine-1,1-dioxide

The title compound is prepared according to the procedure described in Example 33, Step 2, starting from 7,8-dichloro-3-methylbenzo[1,2,3]thiadiazine-3-ium-1-oxide-1-oate (product of Step 1; 2.17 g; 0.0082 mol) and sodium borohydride (1.25 g, 0.0325 mol).

Yield, 2.0 g, white crystals (91%)

Melting point, 189-190° C. (ethanol)

IR (KBr): 3170 (NH); 1329, 1170 (S═O) cm⁻¹.

¹HNMR (DMSO, 400 MHz): 8.65 (1H, s); 7.85 (1H, d, J=8.4 Hz); 7.42 (1H, d, J=8.5 Hz); 4.03 (2H, s), 2.68 (3H, s) ppm.

¹³CNMR (DMSO, 400 MHz): 137.9; 136.4; 133.0; 131.9; 128.4; 127.9; 56.6; 45.7 ppm.

Elemental analysis [calculated on the basis of the Formula C₈H₈Cl₂N₂O₂S (267.14)]:

Calculated: C, 35.97; H, 3.02; Cl 26.54; N 10.49; S 12.00%

Measured: C, 36.29; H, 3.12; Cl 26.61; N 10.57; S 11.72%

### Example 37

### 7-chloro-3-methyl-3,4-dihydrobenzo[1,2,3]thiadiazine-1,1-dioxide

### Step 1

### 7-chloro-3-methylbenzo[1,2,3]thiadiazine-3-ium-1-oxide-1-oate

The title compound is prepared according to the procedure of Example 32, Step 1, starting from 7-chlorobenzo[1,2,3]thiadiazine-1,1-dioxide (3.25 g; 0.015 mol), sodium hydride (50% by weight; 0.79 g; 0.0165 mol) and methyl iodide (2.79 ml; 6.39 g; 0.045 mol).

Yield, 1.07 g, white crystals (31%)

Melting point, 225-227° C. (acetonitrile).

IR (KBr): 1286, 1106 (S═O) cm⁻¹.

¹HNMR (DMSO, 400 MHz): 8.94 (1H, t, J=0.9 Hz); 7.93-7.88 (3H, m); 4.02 (3H, d, J=0.9 Hz) ppm.

¹³CNMR (DMSO, 400 MHz): 139.4; 136.8; 132.0; 131.2; 129.9; 124.9; 119.8; 52.0 ppm.

Elemental analysis [calculated on the basis of the Formula C₈H₇ClN₂O₂S (230.67)]:

Calculated: C, 41.66; H, 3.06; Cl 15.37; N 12.14; S 13.90%

Measured: C, 41.66; H, 2.97; Cl 15.38; N 12.01; S 13.88%

### Step 2

### 7-chloro-3-methyl-3,4-dihydrobenzo[1,2,3]thiadiazine-1,1-dioxide

The title compound is produced according to the procedure of Example 33, Step 2, starting from 7-chloro-3-methylbenzo[1,2,3]thiadiazine-3-ium-1-oxide-1-oate (product of Step 1; 2.30 g; 0.01 mol) and sodium borohydride (1.52 g; 0.04 mol).

Yield, 2.24 g, white crystals (96%)

Melting point, 188-189° C. (ethanol)

IR (KBr): 3165 (NH); 1327, 1172 (S═O) cm⁻¹.

¹HNMR (DMSO, 500 MHz): 8.50 (1H, s); 7.85 (1H, d, J=2.2 Hz); 7.66 (1H, dd, J=2.1; 8.5 Hz); 7.43 (1H, d, J=8.5 Hz); 3.98 (2H, s); 2.69 (3H, s) ppm.

¹³CNMR (DMSO, 500 MHz): 137.8; 134.6; 132.1; 131.9; 129.4; 123.4; 55.5; 45.8 ppm.

Elemental analysis [calculated on the basis of the Formula C₈H₉ClN₂O₂S (232.69)]:

Calculated: C, 41.29; H, 3.90; Cl 15.24; N 12.04; S 13.78%

Measured: C, 41.47; H, 4.01; Cl 15.12; N 12.01; S 13.54%

### Example 38

### 7,8-Dichloro-4,5-dimethyl-3-isopropyl-3,4-dihydrobenzo[1,2,3]thiadiazine-1,1-dioxide

7,8-Dichloro-4,5-dimethylbenzo[1,2,3]thiadiazine-1,1-dioxide (1.95 g; 0.007 mol) is added to acetic acid (35 ml) and acetone (2 ml) and hydrogenated at 10 bar hydrogen pressure in the presence of platinum(IV)-oxide (0.1 g) catalyst. After the calculated amount of hydrogen is used up, the catalyst is filtered off and the filtrate is evaporated. Diethylether (10 ml) is added to the residue and filtered.

Yield, 1.51 g, white crystals (66.7%)

Melting point, 167-168° C. (ethanol)

IR (KBr): 3209 (NH); 1339, 1186 (S═O) cm⁻¹

¹HNMR (DMSO, 200 MHz): 8.62 (1H, s); 7.74 (1H, q, J=0.6 Hz); 4.35 (1H, q, J=6.6 Hz); 3.07 (1H, m); 2.34 (3H, d, J=0.4 Hz); 1.34 (3H, d, J=6.6 Hz); 1.13 (3H, d, J=6.3 Hz); 1.10 (3H, d, J=6.3 Hz) ppm.

¹³CNMR (DMSO, 400 MHz): 141.9; 136.4; 136.0; 134.4; 131.1; 125.6; 53.0; 52.1; 20.3; 18.6; 11.3 ppm.

Elemental analysis [calculated on the basis of the Formula C₁₂H₁₆Cl₂N₂O₂S (323.24)]:

Calculated: C, 44.59; H, 4.99; Cl 21.94; N 8.67; S 9.92%

Measured: C, 44.86; H, 5.02; Cl 21.78; N 8.66; S 9.86%

### Example 39

### 2,3-Dimethyl-6-methoxy-3,4-dihydrobenzo[1,2,3]thiadiazine-1,1-dioxide

The solution of 3-methyl-6-methoxy-3,4-dihydrobenzo-[1,2,3]thiadiazine-1,1-dioxide (compound of Example 34; 1.70 g; 0.0075 mol) prepared in N,N-dimethylformamide (22.5 ml) is added dropwise to the suspension of potassium-tent-butylate (1.68 g; 0.015 mol) prepared in N,N-dimethylformamide (7.5 ml) at room temperature. After 30 minutes stirring, methyl iodide (1.4 ml; 3.19 g; 0.0225 mol) is dropped slowly to the reaction mixture. After further 2-hour stirring, the mixture is poured onto an ice-water mixture. The crystals thus obtained are filtered.

Yield, 1.22 g, white crystals (66%)

Melting point, 142-143° C. (ethanol).

IR (KBr): 1332, 1169 (S═O) cm⁻¹.

¹HNMR (CDCl₃, 400 MHz): 7.80 (1H, d, J=8.8 Hz); 6.95 (1H, dd, J=2.5; 8.7 Hz); 6.67 (1H, d, J=2.6 Hz); 3.92 (2H, s); 3.84 (3H, s); 2.82 (3H, s); 2.74 (3H, s) ppm.

¹³CNMR (CDCl₃, 400 MHz): 161.9; 136.2; 127.7; 125.8; 114.2; 111.0; 55.6; 49.1; 43.1; 27.2 ppm.

Elemental analysis [calculated on the basis of the Formula C₁₀H₁₄N₂O₃S (242.30)]:

Calculated: C, 49.57; H, 5.82; N, 11.56; S 13.23%

Measured: C, 49.16; H, 5.74; N, 11.43; S 13.18%

### Example 40

### 3-Ethyl-2-methyl-6-methoxy-3,4-dihydrobenzo[1,2,3]thiadiazine-1,1-dioxide

The title compound is produced according to the procedure described in Example 39, starting from 3-ethyl-6-methoxy-3,4-dihydrobenzo[1,2,3]thiadiazine-1,1-dioxide (compound of Example 35; 1.70 g; 0.007 mol), potassium-tent-butylate (1.68 g; 0.015 mol) and methyl iodide (1.4 ml; 3.19 g; 0.0225 mol).

Yield, 1.4 g, white crystals (73%).

Melting point, 123-125° C. (ethanol)

IR (KBr): 1322, 1170 (S═O) cm⁻¹.

¹HNMR (CDCl₃, 400 MHz): 7.80 (1H, d, J=8.7 Hz); 6.94 (1H, dd, J=2.5; 8.7 Hz); 6.68 (1H, d, J=2.5 Hz); 3.94 (2H, s); 3.84 (3H, s); 2.90 (2H, q, J=7.1 Hz); 2.70 (3H, s); 1.27 (3H, t, J=7.1 Hz) ppm.

¹³CNMR (CDCl₃, 400 MHz): 161.9; 136.4; 127.7; 126.4; 114.1; 111.0; 55.6; 49.0; 48.2; 27.5; 12.0 ppm.

Elemental analysis [calculated on the basis of the Formula C₁₁H₁₆N₂O₃S (256.33)]:

Calculated: C, 51.54; H, 6.29; N, 10.93; S 12.51%

Measured: C, 51.18; H, 6.30; N, 10.84; S 12.21%

### Example 41

### 2,3-Dimethyl-3,4-dihydrobenzo[1,2,3]thiadiazine-1,1-dioxide

The title compound is produced according to the procedure disclosed in Example 39 starting from 3-methyl-3,4-dihydrobenzo[1,2,3]thiadiazine-1,1-dioxide (compound of Example 32; 2.50 g; 0.0126 mol), potassium-tert-butylate (2.82 g; 0.025 mol) and methyl iodide (1.56 ml; 3.57 g; 0.0252 mol).

Yield, 1.65 g, white crystals (62%)

Melting point, 143-145° C. (ethanol)

IR (KBr): 1324, 1162 (S═O) cm⁻¹

¹HNMR (CDCl₃, 400 MHz): 7.86 (1H, dd, J=1.4; 7.8 Hz); 7.49 (1H, dt, J=1.4; 7.5 Hz); 7.44 (1H, t, J=7.7 Hz); 7.22 (1H, d, J=7.6 Hz); 3.95 (2H, s); 2.82 (3H, s); 2.75 (3H, s) ppm.

¹³CNMR (CDCl₃, 400 MHz): 134.1; 133.6; 131.8; 128.2; 126.3; 125.6; 48.6; 43.0; 27.0 ppm.

Elemental analysis [calculated on the basis of the Formula C₉H₁₂N₂O₂S (212.27)]:

Calculated: C, 50.93; H, 5.70; N, 13.20; S 15.11%

Measured: C, 50.94; H, 5.68; N, 13.14; S 15.11%

### Example 42

### 3-Ethyl-2-methyl-3,4-dihydrobenzo[1,2,3]thiadiazine-1,1-dioxide

The title compound is prepared according to the procedure of Example 39 starting from 3-ethyl-3,4-dihydrobenzo[1,2,3]thiadiazine-1,1-dioxide (compound of Example 33; 2.04 g; 0.0096 mol), potassium-tent-butylate (2.15 g; 0.0192 mol) and methyl iodide (1.75 ml; 3.99 g; 0.028 mol).

Yield, 1.64 g, white crystals (72%)

Melting point, 140-142° C. (ethanol)

IR (KBr): 1386, 1155 (S═O) cm⁻¹.

¹HNMR (CDCl₃, 400 MHz): 7.86 (1H, dd, J=1.4; 7.8 Hz); 7.49 (1H, dt, J=1.5; 7.4 Hz); 7.43 (1H, dt, J=0.7; 7.7 Hz); 7.23 (1H, d, J=7.2 Hz); 3.98 (2H, s); 2.92 (2H, q, J=7.1 Hz); 2.72 (3H, s); 1.27 (3H, t, J=7.1 Hz) ppm.

¹³CNMR (CDCl₃, 400 MHz): 134.2; 131.8; 128.2; 126.4; 125.8; 48.9; 47.8; 27.3; 11.9 ppm.

Elemental analysis [calculated on the basis of the Formula C₁₀H₁₄N₂O₂S (226.30)]:

Calculated: C, 53.08; H, 6.24; N, 12.38; S 14.17%

Measured: C, 52.93; H, 6.25; N, 12.28; S 14.13%

### Example 43

### 7,8-Dichloro-2,3,4-trimethyl-3,4-dihydrobenzo[1,2,3]thiadiazine-1,1-dioxide

The title compound is produced according to the procedure described in Example 39 starting from 7,8-dichloro-2,4-dimethyl-3,4-dihydrobenzo[1,2,3]thiadiazine-1,1-dioxide (compound of Example 16; 1.64 g; 0.0058 mol), paraformaldehyde (1.6 g) and palladium-charcoal catalyst (10% by weight, 0.2 g).

Yield, 1.5 g, white crystals (88%)

Melting point, 135-136° C. (ethanol).

IR (KBr): 1343, 1156 (S═O) cm⁻¹.

¹HNMR (CDCl₃, 400 MHz): 7.58 (1H, d, J=8.5 Hz); 7.17 (1H, d, J=8.5 Hz); 4.05 (1H, q, J=6.0 Hz); 2.87 (3H, s); 2.85 (3H, s); 1.50 (3H, d, J=6.6 Hz) ppm.

Elemental analysis [calculated on the basis of the Formula C₁₀H₁₂Cl₂N₂O₂S (295.19)]:

Calculated: C, 40.69; H, 4.10; Cl 24.02; N 9.49; S 10.86%

Measured: C, 41.12; H, 4.08; Cl 23.88; N 9.45; S 10.83%

1. 3,4-dihydrobenzo[1,2,3]thiadiazine-1,1-dioxide derivatives of the Formula (I) 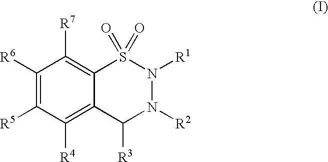
wherein
R1, R2, R3 represent independently hydrogen or a straight- or branched-chain alkyl group having 1 to 4 carbon atoms;
R4, R5, R6 and R7 each represents hydrogen, halogen, a straight- or branched-chain alkyl group having 1 to 4 carbon atoms or an alkoxy group containing a straight- or branched-chain alkyl group having 1 to 4 carbon atoms; or
stereoisomers of the compounds of the Formula (I) and the mixtures thereof.

2. 7,8-Dichloro-2-methyl-3,4-dihydrobenzo[1,2,3]thiadiazine-1,1-dioxide, its stereoisomers and the mixtures thereof as defined in claim 1.

3. 7,8-Dichloro-2,4-dimethyl-3,4-dihydrobenzo[1,2,3]thiadiazine-1,1-dioxide, its stereoisomers and the mixtures thereof as defined in claim 1.

4. 7,8-Dichloro-4,5-dimethyl-3,4-dihydrobenzo[1,2,3]thiadiazine-1,1-dioxide, its stereoisomers and the mixtures thereof as defined in claim 1.

5. 7,8-Dichloro-4-methyl-3,4-dihydrobenzo[1,2,3]thiadiazine-1,1-dioxide, its stereoisomers and the mixtures thereof as defined in claim 1.

6. 7-chloro-4-methyl-3,4-dihydrobenzo[1,2,3]thiadiazine-1,1-dioxide, its stereoisomers and the mixtures thereof as defined in claim 1.

7. Process for the preparation of 3,4-dihydrobenzo[1,2,3]thiadiazine-1,1-dioxide derivatives of the Formula (I) as defined in claim 1, stereoisomers thereof and the mixtures of the stereoisomers, wherein
R1, R2, R3 represent independently hydrogen or a straight- or branched-chain alkyl group comprising 1 to 4 carbon atoms;
R4, R5, R6 and R7 each represents hydrogen, halogen, a straight- or branched-chain alkyl group having 1 to 4 carbon atoms or an alkoxy group containing a straight- or branched-chain alkyl group having 1 to 4 carbon atoms;
which comprises
a) for the preparation of the 3,4-dihydrobenzo[1,2,3]thiadiazine-1,1-dioxide derivatives of the Formula (I), wherein R1 represents hydrogen or a straight- or branched-chain alkyl group having 1 to 4 carbon atoms, R2 represents hydrogen and the meaning of R3, R4, R5, R6,
R7 is as defined in claim 1, reducing a compound of the general Formula (II) 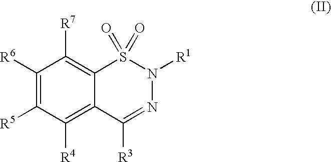
wherein R1 represents hydrogen or a straight- or branched-chain alkyl group and the meaning of R3, R4, R5, R6, R7 is as defined in claim 1, or
b) for the preparation of 3,4-dihydrobenzo[1,2,3]thiadiazine-1,1-dioxide derivatives, wherein R1 represents a straight- or branched-chain alkyl group having 1 to 4 carbon atoms, R2 represents hydrogen and the meaning of R3, R4, R5, R6, R7 is as defined in claim 1, alkylating a compound of the general Formula (I) wherein R1 and R2 represent independently hydrogen and the meaning of R3, R4, R5, R6, R7 is according to claim 1 in an organic solvent in the presence of an acid-binding agent using an alkyl-halogenide containing a straight- or branched-chain alkyl group having 1 to 4 carbon atoms; or
c) for the preparation of 3,4-dihydrobenzo[1,2,3]thiadiazine-1,1-dioxide derivatives, wherein R1 represents hydrogen or a straight- or branched-chain alkyl group having 1 to 4 carbon atoms, R2 represents a straight- or branched-chain alkyl group having 1 to 4 carbon atoms and the meaning of R3, R4, R5, R6, R7 is the same as defined in claim 1, reacting a compound of the Formula (I) wherein R1 is hydrogen or a straight- or branched-chain alkyl group having 1 to 4 carbon atoms, R2 represents hydrogen and the meaning of ‘R3, R4, R5, R6, R7 is the same as defined in claim 1, with an aliphatic aldehyde or ketone having 1 to 4 carbon atoms under the conditions of the reductive alkylation; or
d) for the preparation of 3,4-dihydrobenzo[1,2,3]thiadiazine-1,1-dioxide derivatives of the Formula (I), wherein R1 is hydrogen or a straight- or branched-chain alkyl group having 1 to 4 carbon atoms, R2 represents a straight- or branched-chain alkyl group having 1 to 4 carbon atoms and the meaning of R3, R4, R5, R6, R7 is as defined in claim 1, subjecting a compound of the Formula (II) wherein R1 represents hydrogen or a straight- or branched-chain alkyl group having 1 to 4 carbon atoms and the meaning of R3, R4, R5, R6, R7 is as defined in claim 1 to catalytical reduction and reacting the thus obtained product with an aliphatic aldehyde or ketone having 1 to 4 carbon atoms under the conditions of reductive alkylation; or
e) for the preparation of 3,4-dihydrobenzo[1,2,3]thiadiazine-1,1-dioxide derivatives of the Formula (I), wherein R1 and R3 represent independently hydrogen, R2 represents a straight- or branched-chain alkyl group having 1 to 4 carbon atoms and the meaning of R4, R5, R6, R7 is as defined in claim 1, transforming a compound of the general Formula (III) 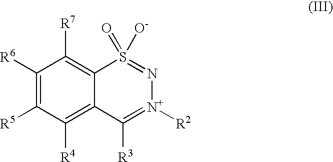
wherein R2 represents a straight- or branched chain alkyl group having 1 to 4 carbon atoms, R3 is hydrogen and the meaning of R4, R5, R6, R7 is as defined in claim 1 by reduction using hydrogen in the presence of platinum(IV)-oxide catalyst or by using sodium borohydride, into a compound of the Formula (I), or
f) for the preparation of 3,4-dihydrobenzo[1,2,3]thiadiazine-1,1-dioxide derivatives of the Formula (I), wherein R1 and R3 are hydrogen, R2 represents a straight- or branched-chain alkyl group having 1 to 4 carbon atoms and the meaning of R4, R5, R6, R7 is the same as in claims 1, reacting a compound of the Formula (II) wherein R1 is hydrogen and the meaning of R2, R3, R4, R5, R6, R7 is as defined in claim 1, with an alkyl halogenide in the presence of sodium hydride in an organic solvent and reducing the thus obtained compound of the Formula (III) wherein R1 represents a straight- or branched-chain alkyl having 1 to 4 carbon atoms and the meaning of R2, R3, R4, R5, R6, R7 is as defined in claim 1 catalytically or by using sodium borohydride, or
g) for the preparation of 3,4-dihydrobenzo[1,2,3]-thiadiazine-1,1-dioxide derivatives of the Formula (I), wherein R1 and R2 independently represent a straight- or branched-chain alkyl group having 1 to 4 carbon atoms and the meaning of R3, R4, R5, R6, R7 is as defined in claim 1, reacting a compound of the Formula (I) wherein R1 is hydrogen, R2 represents a straight- or branched-chain alkyl group having 1 to 4 carbon atoms and the meaning of R3, R4, R5, R6, R7 is as defined in claim 1 with an alkyl halogenide using an organic solvent, preferably N,N-dimethylformamide or tetrahydrofurane in the presence of an acid-binding reagent, preferably, potassium-tert-butylate.

8. Process variant a) of claim 7 characterized in that the reduction is carried out by heterogeneous catalysis in the presence of a noble metal catalyst, preferably, platinum(IV)-oxide under hydrogen overpressure using an organic solvent, preferably acetic acid.

9. Process variant b) of claim 7 characterized in that as acid-binding reagent, an organic or inorganic base, preferably potassium-tert-butylate, sodium hydride or triethylamine is used.

10. Process variant b) of claim 7 characterized in that as organic solvent, preferably a polar aprotic solvent, especially advantageously N,N-dimethylformamide or tetrahydrofurane is used.

11. Process variant c) or d) of claim 7, characterized in that the reductive alkylation is performed in the presence of palladium-charcoal or platinum(IV)-oxide catalyst, in an organic solvent, preferably, in acetic acid or tetrahydrofurane under hydrogen overpressure.

12. (canceled)

13. Medicaments suitable for the treatment of the diseases or disorders of the central nervous system, containing a 3,4-dihydrobenzo[1,2,3]thiadiazine-1,1-dioxide derivative of the general Formula (I), as defined in claim 1 wherein
R1, R2, R3 represent independently hydrogen or a straight- or branched-chain alkyl group having 1 to 4 carbon atoms;
R4, R5, R6 and R7 each represents hydrogen, halogen, a straight- or branched-chain alkyl group having 1 to 4 carbon atoms or an alkoxy group containing a straight- or branched-chain alkyl group having 1 to 4 carbon atoms;
or a stereoisomer of a compound of the Formula (I) or a mixture thereof, one or more pharmaceutically acceptable vehicle and optionally pharmaceutical auxiliary agents.

14. Medicaments suitable for the prevention or treatment for disorders or diseases belonging to the group of anxiety disorders including generalized anxiety disorder, panic disorder, agoraphobia, social phobia, other types of phobias, posttraumatic stress disorder containing a 3,4-dihydrobenzo[1,2,3]thiadiazine-1,1-dioxide derivative of the Formula (I) as defined in claim 1, wherein
R1, R2, R3 represent independently hydrogen or a straight- or branched-chain alkyl group having 1 to 4 carbon atoms;
R4, R5, R6 and R7 each represents hydrogen, halogen, a straight- or branched-chain alkyl group having 1 to 4 carbon atoms or an alkoxy group containing a straight- or branched-chain alkyl group having 1 to 4 carbon atoms;
or a stereoisomer of a compound of the Formula (I) or a mixture thereof, in admixture with one or more pharmaceutically acceptable vehicle(s) and optionally pharmaceutical auxiliary agents.

15. Medicaments suitable for the prevention or treatment of diseases or disorders of the central nervous system exhibiting the symptoms of anxiety, including attention-deficit hyperactivity disorder, stress-related adaptation disorder, posttraumatic stress disorder, anorexia nervosa, bulimia nervosa, insomnia, parasomnia, compulsive disorders including obsessive-compulsive disorders, disturbances of the sexual function and for the treatment or prevention of symptoms occurring due to the use or withdrawal of chemical agents like alcohol, caffeine, drugs of abuse, sedatives, narcotics or doping agents containing a 3,4-dihydrobenzo[1,2,3]thiadiazine-1,1-dioxide derivative of the Formula (I) as defined in claim 1, wherein
R1, R2, R3 represent independently hydrogen or a straight- or branched-chain alkyl group having 1 to 4 carbon atoms;
R4, R5, R6 and R7 each represents hydrogen, halogen, a straight- or branched-chain alkyl group having 1 to 4 carbon atoms or an alkoxy group containing a straight- or branched-chain alkyl group having 1 to 4 carbon atoms;
or a stereoisomer of a compound of the general Formula (I) or a mixture thereof, in admixture with one or more pharmaceutically acceptable vehicle(s) and optionally pharmaceutical auxiliary agents.

16. (canceled)

17. (canceled)

18. (canceled)

19. Method for the treatment or prevention of the disorders of diseases of the central nervous system, especially diseases belonging to the group of anxiety disorders including generalized anxiety disorder, panic disorder, agoraphobia, social phobia, other types of phobias, posttraumatic stress disorder and for those diseases or disorders of the central nervous system which are accompanied by the symptoms of anxiety including attention-deficit hyperactivity disorder, stress-related adaptation disorder, posttraumatic stress disorder, anorexia nervosa, bulimia nervosa, insomnia, parasomnia, compulsive disorders including obsessive-compulsive disorders, disturbances of the sexual function and the symptoms manifesting due to the use or withdrawal of chemical agents including alcohol, caffeine, nicotine, sedatives, narcotics, doping agents or drugs of abuse, which comprises administering to a person in need of such treatment a therapeutically effective dose of a 3,4-dihydrobenzo[1,2,3]thiadiazine-1,1-dioxide derivative of the Formula (I) according to claim 1, a stereoisomer thereof or the mixture of the stereoisomers.
